SEQUENCE LISTING (4 sequences)

SEQ ID NO: 1 - protein, 329 aa, Artificial Sequence
  IgG1 HC constant region
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 8 aa, Artificial Sequence
  p15 peptide
KSPWFTTL

SEQ ID NO: 3 - protein, 7 aa, Artificial Sequence
  OVA 257-264 peptide (OVA-1)
IINFEKL

SEQ ID NO: 4 - protein, 16 aa, Artificial Sequence
  OVA 265-280 peptide (OVA-2)
TEWTSSNVMEERKIKV

(19) United States
(12) Patent Application Publication
(10) Pub. No.: US 2024/0423927 A1
(43) Pub. Date: Dec. 26, 2024

(54) COMPOSITIONS AND METHODS FOR USE IN IMMUNOTHERAPY

(71) Applicants: INSTITUT CURIE, PARIS (FR); INSERM (INSTITUT NATIONAL DE LA SANTÉ ET DE LA RECHERCHE MÉDICALE), PARIS (FR); STIMUNITY, PARIS (FR)

(72) Inventors: NICOLAS MANEL, PARIS (FR); BAKHOS JNEID, SAINT-CYR-L'ECOLE (FR)

(21) Appl. No.: 18/287,664

(22) PCT Filed: Apr. 20, 2022
(86) PCT No.: PCT/EP2022/060422

§ 371 Date: Oct. 20, 2023

(30) Foreign Application Priority Data
Apr. 20, 2021 (EP) 21305522.1

(51) Int. Cl. A61K 9/51; A61K 31/506; A61K 31/7084; A61K 39/00; A61P 35/00; C07K 16/28; C12N 15/86

(52) U.S. Cl. CPC A61K 9/5184; A61K 31/506; A61K 31/7084; A61P 35/00; C07K 16/2818; C07K 16/2866; C12N 15/86; A61K 2039/505; C12N 2740/16211; C12N 2760/20223

(57) ABSTRACT

The present invention relates to a pharmaceutical combination comprising i) a vector comprising a stimulator of interferon genes (STING) activator and ii) an anti-regulatory T cells (Tregs) agent, and to uses thereof, in particular for the treatment of cancer or of a STING-mediated disease or disorder.

### CROSS-REFERENCE TO RELATED APPLICATION

This application is the U.S. national stage application of International Patent Application No. PCT/EP2022/060422, filed Apr. 20, 2022.

### REFERENCE TO SEQUENCE LISTING

The Sequence Listing for this application is labeled “Seq-List-replace.txt” which was created on Apr. 27, 2024 and is 3,913 bytes. The entire content of the sequence listing is incorporated herein by reference in its entirety.

### FIELD OF THE INVENTION

The present invention relates to immunotherapy, in particular to compositions and methods of enhancing a host's immune response, more specifically to compositions and methods for inducing effector T cells and blocking the ability of regulatory T cells, preferably intratumoral regulatory T cells, to suppress host immune responses.

### BACKGROUND OF THE INVENTION

There are three main stages in the development of the tumor. During the initial stage, immune cells eliminate continuously arising transformed cells thanks to interferon-γ (IFN-γ) production and lymphocyte effector function. The second stage of tumor development is an equilibrium phase of dynamic balance where IFN-γ production and lymphocyte effector function relentlessly attack tumor cells thereby prohibiting tumor growth but are unable to eradicate transformed cells. This stage of equilibrium allows for the development of tumor heterogeneity and genetic instability in cells that survive elimination. The final stage of tumor development is escape. Tumor cell variants that were selected for during the equilibrium phase are now able to grow unchecked even in the presence of a competent immune system (Dunn et al., 2004, Immunity, Vol. 21, 137-148).

Tumors employ multiple mechanisms for avoiding immune elimination including down-regulation of positive signals to tumor specific CD8⁺ cytotoxic T cells (CTLs) and the accumulation of regulatory T (Treg) cells in the tumor microenvironment (TME), i.e., in and around the tumor. It is now well established that Tregs are a suppressive subset of CD4⁺ T cells endowed with regulatory properties that affect a variety of immune cells such as effectors CD4⁺ and CD8⁺ and natural killer (NK) cells and inhibit dendritic cell activation. Functionally, Tregs are capable of inhibiting the proliferation and killing activity of CTLs through several mechanisms such as secretion of cytokines (TGF-β1) and IL-10, metabolic disruption through CD39 and CD73 (Deaglio et al., 2007, J Exp Med. Vol. 204(6):1257-65), or contact-dependent inhibition via programmed death ligand 1 (PD-L1) signaling (Wu et al., 2018, Oncoimmunology, Vol. 7, n°11, e1500107). High tumor infiltration by Tregs and a low ratio of effector T cells (Teffs) to Tregs is associated with poor outcome in solid tumors.

Tregs are characterized by their expression of the high affinity IL-2 receptor, CD25, and the transcription factor forkhead box P3 (Foxp3) (Shimon Sakaguchi et al., 2008, Eur. J. Immunol. 38: 901-937). Foxp3 is a master regulator in Treg cells and is essential for their development and suppressive function (Maruyama et al., 2011, Semin. Immunol. Vol. 23(6):418-23). Treg expansion observed during tumor progression may result from the proliferation of naturally occurring Tregs (nTregs) or from conversion of CD4⁺CD25⁻FoxP3⁻ T cells into CD4⁺CD25⁺FoxP3⁺ Tregs (iTregs) in the presence of IL-2 and TGF-b1. Though identical in their suppressive function, these cells differ in their stability of Foxp3. In nTREGS, Foxp3 expression is highly stable and constitutively expressed whereas in iTREGS, such as those induced at tumor sites, Foxp3 expression is unstable (Floess et al. 2007, PLoS Biol. 2007 Feb; 5(2): e38). Measurement of Foxp3 transcript level in the tumors provides no clear evidence of the amount of Tregs in the tumor microenvironment. Tumor heterogeneity is a first obstacle that impede the success of cancer immunotherapies.

In many types of cancer, upregulation of immune checkpoint molecules such as programmed death 1 (PD-1) and other inhibitory receptors such as T cell immunoglobulin mucin 3 (TIM-3), cytotoxic T lymphocyte antigen-4 (CTLA-4), glucocorticoid-induced tumor necrosis factor receptor (GITR), and lymphocyte activation gene-3 (LAG-3) occur in tumor infiltrating Treg cells (Park et al., 2012, Cell Immunol., 278(1-2):76-83). T cell Ig and ITIM domain (TIGIT), another immune checkpoint is also present in Treg (Kim et al., 2019, Journal for ImmunoTherapy of Cancer 7:339).

Because Tregs are one of the main barriers for the eradication of tumors by immune cells, their therapeutic depletion or their functional inactivation using drugs or antibodies improves responses to cancer immunotherapy. Several receptors and enzymes expressed on Tregs have been identified as potential targets to enhance antitumor immunity (Sundee et al., 2021, Eur. J. Immunol., 51:280-291). However, due to the expression of some of these receptors on other immune cells, including effector T cells and NK cells, the relevance of their use as immunotherapeutic treatment of cancer is still under debate. Moreover, it should be kept in mind that proper number and functions of regulatory T cells (Treg), in particular intratumoral regulatory T cells, are essential for a well-balanced immune system: too few of these cells leads to autoimmunity and too much prevents an efficient immune response, with harmful consequences for anti-tumor immunity, for instance.

Clinical studies exploring the use of vaccines in combination with daclizumab, a humanized IgG1 anti-human CD25 antibody, or denileukin diftitox, a recombinant fusion protein combining human IL-2 and a fragment of diphtheria toxin, or LMB-2, a recombinant fusion protein combining anti-human CD25 Fv and a fragment of Pseudomonas exotoxin A (PE) had a variable impact on the number of circulating Tregs and vaccine-induced immunity (Luke et al., 2016, Journal for ImmunoTherapy of Cancer 4:35/Jacobs et al., 2010, Clin Cancer Res 16:5067-5078/Powell et al., 2007, J Immunol., 179(7): 4919-4928). Moreover, the selective elimination or inactivation in the tumor of Tregs using anti-CD25 antibody remains a major challenge because these cells share the same surface markers (CD25) as activated conventional, non-suppressive T cells. A complete depletion in Treg may greatly impair the self-tolerance mechanism. Consequently, systemic depletion of Tregs may not be a good choice for cancer treatment.

Another approach relies on the use of immune checkpoint blocking (ICB) antibodies to block the binding of inhibitory molecules and boost the antitumor immune response. Currently, there are several FDA approved ICB antibodies on the market against CTLA-4 (Ipilimumab), PD-1 (Pembrolizumab, Nivolumab and Cemiplimab) and PD-L1 (Atezolizumab, Avelumab and Durvalumab). Despite major advances in immunotherapy, the clinical use of ICB antibodies is limited to a small number of cancer types (C. Lee Ventola, 2017, P&T®, Vol. 42 No. 8). Moreover, acquired resistance to ICB antibodies has revealed the need for additional treatments.

The stimulator of interferon genes (STING) protein is a transmembrane receptor localized to the endoplasmic reticulum that recognizes and binds cyclic dinucleotides. Adjuvant formulations comprising a STING agonist capable of inducing an immune response in a subject are disclosed in WO2019136118. These formulations, when used with a means for inducing antigen release from a cell, enhance the immunogenicity of released antigens from cells during treatment. Another option to increase immune activity in the treatment of cancer is to use the combination of a STING agonist and of a purinergic receptor agonist (WO2020/227159). Other STING agonists, in particular compound no. 14, used in combination with a checkpoint inhibitor, provide a beneficial effect during a few days in several mouse syngeneic tumor models exposed to the combination (WO2021/005541). However, recent studies on the contrary suggest a potential inhibitory effect of STING activation on adaptive antitumor immune responses, notably by preventing T cells proliferation and promoting their death. Moreover, non-immune functions of STING have been described as facilitating the survival and growth of metastatic cancer cells (Zhu et al., 2019, Molecular Cancer, 18: 152).

Thus, there is in particular a strong need for therapeutic agents and methods for use for optimizing the manipulation of the immune suppressive action of Treg cells, in particular for use for improving the treatment of cancer.

### SUMMARY OF THE INVENTION

The present invention is based, at least in part, on the discovery that combining an activation of the stimulator of interferon genes (STING) pathway with a modulation, preferably an inhibition, of the regulatory T cells (Tregs) subpopulations, even more preferably of intratumoral regulatory T cells, makes it possible to significantly and very advantageously improve the treatment of cancer.

The present invention relates to a composition/combination comprising i) a STING (stimulator of interferon genes) activator, preferably in a vectorized form, i.e., a vector comprising (containing or expressing) a STING activator, in other words a vectorized STING activator, and ii) an anti-regulatory T cells (Tregs) agent, preferably an anti-intratumoral Tregs agent, selected from imatinib or a derivative or salt thereof; dasatinib or a derivative or salt thereof, an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof.

The composition/combination herein described by inventors is suitable both in vitro, for experimental purposes, and in vivo, for therapeutic purposes. The present invention in particular relates to a pharmaceutical composition/combination, for example a therapeutic, vaccine or veterinary composition/combination, comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, selected from imatinib or a derivative or salt thereof, dasatinib or a derivative or salt thereof, an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof.

Preferably, the STING activator is selected from a small molecule, in particular cyclic dinucleotides; a nucleic acid coding for a STING agonist, a vector or cell expressing STING or a STING agonist; an antibody conjugated STING agonist; and an inhibitor of ectonucleotide pyrophosphatase phosphodiesterase 1 (ENPP1).

Preferably, the cyclic dinucleotides is cyclic guanosine monophosphate-adenosine monophosphate (cGAMP), cyclic dimeric guanosine monophosphate (c-di-GMP), cyclic dimeric adenosine monophosphate (c-di-AMP) or a vectorized form thereof. More preferably, the cGAMP cyclic dinucleotides is 2′-3′-cyclic GMP-AMP or 3′-3′-cyclic GMP-AMP or a mixture thereof.

Preferably, the vector comprising a STING activator is a vesicle, in particular a liposome; an exosome such as exoSTING™; a virus for example an adenovirus or an oncolytic virus; a virus-like particle (VLP); a polymer; or a hydrogel.

In a particular aspect, the STING activator is a vectorized dinucleotides and the vectorized dinucleotides is a virus-like particle (VLP) comprising a lipoprotein envelope including a viral fusogenic glycoprotein, and containing cyclic guanosine monophosphate-adenosine monophosphate (cGAMP), the cGAMP being packaged into said virus-like particle.

In another aspect, the STING activator is a small molecule and the small molecule is selected from dithio-(RP, RP)-[cyclic[A(2′,5′)pA(3′,5′)p]] (ML RR-S2 CDA, also named ADU-S100); MK-1454; 5,6-dimethyl-xanthenone acetic acid (DMXAA); Flavone-8-acetic acid (FAA); 10-carboxymethyl-9-acridanone (CMA); α-Mangostin (xanthone); ML RR-S2 CDG; ML RR-S2 cGAMP; 6-bromo-N-(naphthalen-1-yl)benzo[d][1,3]dioxole-5-carboxamide (BNBC); dispiro diketopiperzine (DSDP); the bis-phosphothioate analog of 3 2′3′cGAMP named 2′3′-cG^{S}A^{S}MP; a macrocycle-bridged STING agonist (MBSA); the MBSA derivative of ADU-S100 named E7766; a benzothiophene oxobutanoic acid (MSA-2) derivative; SB11285; diamidobenzimidazole (diABZI); IMSA-101, TAK-676; CRD5500; TTI-10001; and SEL312-4787.

In a preferred aspect, the anti-Tregs agent is an anti-CTLA-4 antibody having an IgG2 constant region such as tremelimumab (CP-675,206).

In another preferred aspect, the anti-regulatory T cells (Tregs) agent is an anti-CTLA-4 antibody having mutated IgG1 constant region such as BMS 986249 (or also named CTLA4-Probody), BMS-986288 (or also named CTLA4-NF), Zalifrelimab (or also referred as AGEN1884), Quavonlimab (or also referred as MK-1308), HBM4003 (from Harbour BioMed) or ONC-392.

In another preferred aspect, the anti-regulatory T cells (Tregs) agent, preferably the anti-intratumoral Tregs agent, is an anti-CTLA-4 antibody wherein the mutation of the IgG1 constant region is located in a CH2 domain of the (Fc) constant region.

In a further preferred aspect, the anti-Tregs agent, preferably the anti-intratumoral Tregs agent, is an anti-CTLA-4 antibody wherein the mutation of the IgG1 constant region is located in a CH3 domain of the (Fc) constant region.

In another preferred aspect, the anti-Tregs agent, preferably the anti-intratumoral Tregs agent, is an anti-CTLA-4 antibody wherein the mutation of the IgG1 constant region is located in a CH1 domain of a Fab region.

In an aspect, the anti-intratumoral anti-regulatory T cells (Tregs) agent is imatinib or a derivative or salt thereof.

In another aspect, the anti-intratumoral anti-regulatory T cells (Tregs) agent is dasatinib or a derivative or salt thereof.

In a particular aspect, a VLP containing a STING activator, imatinib and an immune checkpoint inhibitor are used in combination, in particular in a composition of the invention.

In another particular aspect, a VLP containing a STING activator, an anti-CTLA4 antibody having an IgG2 constant region or a mutated IgG1 constant region and an immune checkpoint inhibitor are used in combination, in particular in a composition of the invention.

In again another particular aspect, a VLP containing a STING activator, an anti-CD25 antibody, in particular an anti-CD25 antibody having an IgG2 constant region or a mutated IgG1 constant region, and an immune checkpoint inhibitor are used in combination, in particular in a composition of the invention.

The present invention also relates to a pharmaceutical composition/combination as herein described, in particular a therapeutic, vaccine or veterinary composition/combination, for use as a medicament. It relates in particular to such a composition/combination for use in prevention or treatment of cancer or of a STING-mediated disease or disorder, preferably cancer, in a subject.

The present invention also relates to a method for treating cancer or a STING-mediated disease or disorder in a subject, in particular cancer, or for preventing cancer or a STING-mediated disease or disorder in a subject, in particular for preventing cancer relapse. This method comprises a step of administering a therapeutically effective amount of a composition, in particular a pharmaceutical, vaccine or veterinary composition, as disclosed herein, to a subject in need thereof. It relates in particular to the use of a composition, in particular a pharmaceutical, vaccine or veterinary composition, as disclosed herein for the manufacture of a medicament or a vaccine for preventing or treating a cancer in a subject. The present invention also relates to a pharmaceutical, vaccine or veterinary composition/combination as disclosed herein for use for preventing or treating cancer, in particular for preventing cancer relapse.

In another aspect, the present invention relates to a method for inducing or stimulating a therapeutic immune effect in a subject in need thereof. The method includes a step of decreasing or inhibiting the immunosuppressive activity of Treg cells by administering a pharmaceutical composition comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, as herein described to a subject in need thereof, wherein decreasing or inhibiting the activity of the Treg cells, in particular intratumoral Treg cells, induces in the subject a therapeutic effect against the disease the subject is suffering of, typically a cancer.

In a further aspect, the present invention relates to a method of decreasing or inhibiting the immunosuppressive functions in a subject. The method includes a step of decreasing or inhibiting the immunosuppressive activity of Treg cells by administering a pharmaceutical composition comprising i) a STING (stimulator of interferon genes) activator, preferably in a vectorized form, i.e., a vector comprising (containing or expressing) a STING activator, and ii) an anti-regulatory T cells (Tregs) agent as herein described, preferably an anti-intratumoral Tregs agent, to a subject in need thereof, wherein decreasing or inhibiting the activity of the Treg cells decreases or inhibit the immunosuppressive functions in the subject.

Preferably, the STING activator is to be administered to the subject by systemic route, in particular subcutaneous (s.c.), oral, intraperitoneal (i.p.), intramuscular (i.m.), intradermal (i.d.), or intravenous (i.v.) route, preferably subcutaneous route.

Preferably, the anti-Tregs agent is to be administered to the subject by systemic route, in particular intraperitoneal, oral, subcutaneous or intravenous route.

The present invention also relates to a kit comprising at least two parts, wherein the first part comprises a STING (stimulator of interferon genes) activator, preferably in a vectorized form, i.e., a vector comprising (containing or expressing) a STING activator, and the second part comprises an anti-regulatory T cells (Tregs) agent, preferably an anti-intratumoral Tregs agent, the first and second parts of the kit being preferably in distinct compartments or containers.

In a particular aspect, the first part of the kit comprises a STING (stimulator of interferon genes) activator in a vectorized form, in other words a vector comprising (containing or expressing) a STING activator, which is a virus-like particle (VLP) and the second part of the kit comprises an anti-regulatory T cells (Tregs) agent, preferably an anti-intratumoral Tregs agent, which is imatinib or a derivative or salt thereof, or an anti-CTLA-4 having an IgG2 constant region or a mutated IgG1 constant region.

The present invention also relates to the use of a composition or of a kit as herein described for the manufacture of a medicament for preventing or treating cancer or a STING-mediated disease or disorder in a subject, preferably cancer.

### BRIEF DESCRIPTION OF THE FIGURES

FIG. 1: Summary of the experimental design to study the effects of cGAMP-VLP in combination with the Imatinib chemotherapeutic agent in MCA-OVA tumor model. Six days post MCA-OVA tumor cell injection (s.c.), mice were randomized into four treatment groups: (1) PBS+PBS; (2) PBS+Imatinib 10 mg/kg; (3) cGAMP-VLP (50 ng cGAMP)+PBS and (4) cGAMP-VLP (50 ng cGAMP)+Imatinib 10 mg/kg. cGAMP-VLP were administered via subcutaneous (s.c.) route and the Imatinib via intraperitoneal (i.p.) route.

FIG. 2: Serum inflammatory cytokines levels (pg/mL) in MCA-OVA tumor-bearing mice treated with cGAMP-VLP or vehicle (PBS) at day 6. Serum samples on day 6, three hours post-treatment, were collected from separate groups of mice following the s.c. injection.

FIG. 3: Specific CD8 and CD4 T cell responses. Ten days after the first s.c. injection of cGAMP-VLP, peripheral blood mononuclear cells (PBMCs) were stimulated with OVA and p15 peptides and assessed by IFN-γ ELISPOT.

FIG. 4: Mean of the tumor growth (cm³) over time with the different treatments indicated on the figure.

FIG. 5: Mean of the tumor growth (cm³) at day 21 with the different treatments indicated on the figure.

FIG. 6: Survival curve of all groups. Death event is defined as tumor size >2 cm³. Statistics were calculated using the log-rank (Mantel-Cox) test.

FIG. 7: Percentage of the body weight loss of mice during the Imatinib treatment.

FIG. 8: Summary of the experimental design to study the effects of cGAMP-VLP in combination with the anti-Tregs agent CTLA-4-mIgG2a in MCA-OVA tumor model. Six days post MCA-OVA tumor cell injection (s.c.), mice were randomized into four treatment groups: (1) PBS+anti-IgG2a isotype; (2) PBS+anti-CTLA4-mIgG2a; (3) cGAMP-VLP (50 ng 15 cGAMP)+anti-IgG2a isotype and (4) cGAMP-VLP (50 ng cGAMP)+anti-CTLA4-mIgG2a. cGAMP-VLP were administered via subcutaneous (s.c.) route and the antibodies via intraperitoneal (i.p.) route.

FIG. 9: Serum inflammatory cytokines levels (pg/mL) in MCA-OVA tumor-bearing mice treated with cGAMP-VLP, and/or anti-CTLA4-mIgG2a or vehicle (PBS) at day 6. Serum samples on day 6, three hours post-treatment, were collected from separate groups of mice following the s.c. injection.

FIGS. 10 and 11: Representative plots for measurement of the percentage and number of Tregs by flow cytometry in MCA-OVA tumors, blood and spleen following the i.p. injection of anti-CTLA4-mIgG2a and the isotype.

FIG. 12: Percentage and number of FOXP3⁺ Treg cells of total CD45⁺ TCRb⁺CD4⁺ T cells with the CD4⁺/Treg and CD8⁺/Treg cell ratios in tumor samples (MCA-OVA tumors), 48 hours after the i.p. injection of antibodies.

FIG. 13: Percentage of FOXP3⁺ Treg cells of total CD45⁺ TCRb⁺CD4⁺ T cells with the CD4⁺/Treg and CD8⁺/Treg cell ratios in blood, 48 hours after the i.p. antibodies injection.

FIG. 14: Percentage of FOXP3⁺ Treg cells of total CD45⁺ TCRb⁺CD4⁺ T cells with the CD4⁺/Treg and CD8⁺/Treg cell ratios in spleen, 48 hours after the i.p. antibodies injection.

FIGS. 15 and 16: Specific CD8 and CD4 T cell responses. Ten days after the first s.c. injection of cGAMP-VLP with or without anti-CTLA4-mIgG2a, peripheral blood mononuclear cells (PBMCs) were stimulated with OVA and p15 peptides and assessed by IFN-γ ELISPOT.

FIG. 17: Measurement of the tumor size (cm³) with a caliper. Every line represents an individual C57BL/6J mouse.

FIG. 18: Mean of the tumor growth over time with the different treatments indicated on the figure and in Example No. 2.

FIG. 19: Survival curve of all groups. Death event is defined as tumor size >2 cm³.

Statistics were calculated using the log-rank (Mantel-Cox) test.

FIG. 20: Tumor volume of tumor-bearing and tumor-free mice after MCA-OVA re-challenge (s.c.) on day 95. CR, complete responder mouse.

FIG. 21: Summary of the experimental design to study the effects of cGAMP-VLP in combination with anti-Tregs agent CD25-mIgG2a (KLC) in MCA-OVA tumor model. Six days post MCA-OVA tumor cell injection (s.c.), mice were randomized into four treatment groups: (1) PBS+anti-IgG2a isotype; (2) PBS+anti-CD25-mIgG2a (KLC); (3) cGAMP-VLP (50 ng cGAMP)+anti-IgG2a isotype and (4) cGAMP-VLP (50 ng cGAMP)+anti-CD25-mIgG2a (KLC). cGAMP-VLP were administered via subcutaneous (s.c.) route and the antibodies via intraperitoneal (i.p.) route.

FIG. 22: Percentage of FOXP3⁺ cells of total TCRb⁺CD4⁺ T cells in splenocytes and tumor samples, 48 hours after the i.p. injection of antibodies.

FIG. 23: Specific CD4 and CD8 T cell responses. Ten days after the first s.c. injection of cGAMP-VLP with or without anti-CD25-mIgG2a, peripheral blood mononuclear cells (PBMCs) were stimulated with two OVA peptides and assessed by IFN-γ ELISPOT.

FIG. 24: Mean of the tumor growth (cm³) over time with the different treatments indicated on the figure and in Example No. 3.

### DETAILED DESCRIPTION OF THE INVENTION

The inventors have developed compositions/combinations that can be used to prevent, alleviate or treat cancer or a STING-mediated disease or disorder, and more generally a disease or condition in which regulatory T cells are blocking the immune response of effector T cells (such as in particular cancer). In particular, the compositions herein described for the first time by the inventors enable a complete regression of the tumors and induce a systemic antitumor immunity in the treated subject.

In a first aspect, the present invention relates to a composition/combination, typically a pharmaceutical composition/combination such as a therapeutic, vaccine or veterinary composition, comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, selected from imatinib or a derivative or salt thereof; dasatinib or a derivative or salt thereof; an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof.

For example, the composition/combination of the invention comprises i) a STING (stimulator of interferon genes) activator, preferably in a vectorized form, i.e., a vector comprising (containing or expressing) a STING activator, and ii) imatinib or a derivative or salt thereof, an anti-CTLA-4 antibody having an IgG2 constant region or a mutated IgG1 constant region, and/or an anti-CD25 antibody, or any other combination of the anti-regulatory T cells (Tregs) agents described in the previous paragraph.

The herein described compositions/combinations are also herein considered as “medicaments”.

The expressions “STING (stimulator of interferon genes)” and “stimulator of interferon genes (STING)” designate the adapter transmembrane protein that is located in the endoplasmic reticulum. STING, also known as TMEM173, MITA, ERIS, and MPYS, is a central signaling molecule in the innate immune response to cytosolic nucleic acids. Human and murine STING amino acid sequences can be found in NCBI Locus NP_938023 and NP_082537 respectively.

STING is activated when double stranded DNA gains access to the cytosol. Beyond its role in sensing the presence of infectious agents (virus, bacteria, parasites and fungi), the STING pathway is also involved in sensing mammalian DNA directly. Cytosolic DNA is detected upon binding to the sensor cyclic-GMP-AMP synthase (cGAS, MB21D1) which catalyzes the synthesis of cyclic GMP-AMP (cGAMP) from guanosine triphosphate (GTP) and adenosine triphosphate (ATP). cGAMP functions as a second messenger that binds and activates STING. Upon binding of cGAMP, STING undergoes conformational changes that trigger its trafficking from the endoplasmic reticulum (ER) to the Golgi to perinuclear endosomes. Consequently, STING recruits tank-binding kinase 1 (TBK1) and is, in turn, phosphorylated by TBK1 which renders it accessible for the binding of the transcription factor interferon regulatory factor 3 (IRF3). TBK1 then phosphorylates IRF3 which translocates to the nucleus to drive transcription of IFN-β and other genes (Corrales and Gajewski, 2015, Clin Cancer Res., 21(21): 4774-4779).

By “STING activator” is meant any natural or synthetic compound that binds to STING and act as an inducer, agonist or enhancer to induce or stimulate the expression of type 1 interferons and other cytokines on incubation with human PBMCs. This binding involves the cGAS-STING signaling pathway. As such, compounds which induce or stimulate the expression of human interferons may be useful in the prevention or treatment of various diseases or disorders, such as pre-cancerous syndromes and cancer. For example, when the STING activator activates STING in the tumor microenvironment (TME), i.e., in and around the tumor, it results in efficient cross-priming of tumor specific antigens to CD8⁺ T cells and facilitates the trafficking of effector T cells by inducing the production of key chemokines, such as, for example, interferon-γ (IFN-γ).

STING activator activity can be determined by one or more STING agonist assays selected from an interferon stimulation assay, a hSTING wt assay, a THPl-Dual assay, a TANK binding kinase 1 (TBK1) assay, and an interferon-γ-inducible protein 10 (IP-10) secretion assay.

STING activators, also known as STING agonists, can be classified into three classes based on their chemical scaffolds and their ability to enter and bind specific amino acids withing the ligand-binding pocket of the cytosolic domain of STING (Feng et al. 2020, Drug Discovery Today, vol. 25(1), 230-237). Cyclic dinucleotides (CDNs) were identified as the first class of molecules capable of binding and activating STING. The second class of molecules that directly connect with the ligand-binding domain (LBD) of STING and activates the cGAS-STING pathway are flavonoids and xanthone derivatives. The third class of STING agonists encompasses diaminobenzimidazoles (diABZIs). Compounds of the three classes of STING agonists, together with CDNs-unrelated agents activating cGAS-STING pathway, can be grouped and are herein identified as “small molecules”.

In a particular aspect, the STING activator is selected from a small molecule, in particular a cyclic dinucleotides; a nucleic acid coding for a STING agonist; a vector or cell comprising/containing/expressing STING or a STING agonist; an antibody conjugated STING agonist such as, for example, SB111325/11396; and an inhibitor of ectonucleotide pyrophosphatase phosphodiesterase 1 (ENPP1) such as, for example, MAVU-104.

The terms “cyclic dinucleotides” refers to small molecule second messengers able to directly bind the endoplasmic reticulum-resident receptor STING (stimulator of interferon genes) and to activate a signaling pathway that induces the expression of type I interferon and also nuclear factor-kB (NFkB) dependent inflammatory cytokines. The natural cyclic dinucleotides can be cyclic guanosine monophosphate-adenosine monophosphate (cGAMP), more specifically c[G(2′,5′)pA(3′,5′)p] (CAS number: 1441190-66-4) or c[G(3′,5′)pA(3′,5′)p](CAS number: 849214-04-6), cyclic dimeric guanosine monophosphate (c-di-GMP), more specifically bis-(3′-5′)-cyclic dimeric guanosine monophosphate (3′,5′-cyclic diguanylic acid, cyclic di-GMP, c-di-GMP), or cyclic dimeric adenosine monophosphate (c-di-AMP), more specifically bis-(3′-5′)-cyclic dimeric adenosine monophosphate (3′,5′-cyclic diadenylic acid, cyclic-di-AMP, c-di-AMP).

In a preferred aspect, the cGAMP cyclic dinucleotides used in the compositions/combinations of the invention is 2′-3′-cyclic GMP-AMP. In another preferred aspect, the cGAMP cyclic dinucleotides used in the compositions/combinations of the invention is 3′-3′-cyclic GMP-AMP.

Among other cyclic dinucleotides suitable for incorporation into a composition/combination of the invention, are derivatives of natural CDNs. ADU-S100 (also known as MIW815 or ML RR-S2 CDA), a dithio derivative of natural CDN 2′-3′ cAMP, and E7766, a macrocycle-bridged derivative of AD-S100, are the first studied synthetic CDNs.

Other non-natural CDNs are currently under development such as, for example, MK1454, SB11285 and BMS-986301.

International Patent Applications WO2014/093936, WO2014/189805, WO2013/185052, WO2015/077354, WO2015/185565 and US2014/0341976 provide examples of cyclic di-nucleotides.

The STING activator, such as for example cyclic dinucleotides, can be used, in the context of the present invention, in a vectorized form.

The vector, for example the vector containing the cyclic dinucleotides or expressing a STING activator or agonist, can be a vesicle, in particular a liposome; an exosome; a virus for example an adenovirus or an oncolytic virus; a virus-like particle (VLP); a polymer; or a hydrogel. Preferably, the vector is a virus or a virus-like particle (VLP), even more preferably, a VLP.

Commercially available exosome suitable in the context of the present invention can be for example exoSTING™ (CODIAK, Cambridge, MA 02140). exoSTING™ is composed of exosomes engineered to express high levels of PTGFRN and an exosomal protein (on the surface of the exosome to facilitate specific uptake in tumor-resident antigen presenting cells), and loaded with a STING agonist (located inside the lumen of the exosome).

Together with liposomes, polymers and hydrogels are main drug delivery systems used nowadays to deliver STING agonists.

Polymers and more particularly polymeric nanoparticles are suitable nanocarriers for STING agonists given their favorable properties including hydrolytic degradability in vivo, controlled drug loading and release kinetics, and overall safety. Polymers usable in the context of the present invention can be selected in the group comprising poly(beta-amino ester) (PBAE), poly(ethylene glycol)-block-[(2-diethylaminoethylmethacrylate)-co-(butyl methacrylate)-co-(pyridyl disulphide ethyl methacrylate)] (PEG-DBP) and Acetylated dextran (Ace-DEX).

Hydrogels are highly hydrophilic polymer networks, which facilitate local and controlled drug release, leading to the recruitment of tumor toxic immune cells. A hydrogel usable in the context of the present invention can be selected in the group comprising linear polyethyleneimine (LPEI)/hyaluronic acid (HA), HA hydrogel scaffold, Matrigel and STINGel.

A virus usable as a vector in the context of the invention can be an adenovirus or an oncolytic virus such as for example a virus derived from herpes simplex-1 virus, vesicular stomatitis virus (VSV) or Newcastle disease virus (NDV).

In a preferred aspect, the STING activator used in the composition of the invention is a vectorized dinucleotides and the vectorized dinucleotides is a virus-like particle (VLP), the dinucleotides being packaged into said virus-like particle and the virus-like particle comprising a lipoprotein envelope including a viral fusogenic glycoprotein. In a particular aspect, the VLP comprises a lipoprotein envelope including a viral fusogenic glycoprotein, and contains cyclic guanosine monophosphate-adenosine monophosphate (cGAMP), the cGAMP being packaged into said virus-like particle.

VLP or Virus-like particle resembles viruses but are non-infectious. It does not contain any wild-type viral genetic material and more preferably any viral infectious genetic material. The expression of viral structural proteins such as envelope or capsid, results in the self-assembly of VLP. VLP can be a virosome (i.e., a lipoprotein envelope devoid of capsid) or a VLP comprising both a capsid and a lipoprotein envelope. The VLP may further comprise an epitope, an antigen or any other protein or nucleic acid of interest, preferably a tumor associated antigen, or a combination thereof.

The enveloped VLPs may include several, in particular two or more, different epitopes/antigens which may be selected (a) different viral strains of the same virus, (b) different serotypes of the same virus, and/or (c) different viral strains specific for different hosts. Different viral strains are, for example, different strains of influenza virus, for example influenza virus A strains H1N1, H5N1, H9N1, H1N2, H2N2, H3N2 and/or H9N2, influenza virus B and/or influenza virus C. Different serotypes are, for example, different serotypes of human papilloma virus (HPV), for example serotypes 6, 11, 16, 18, 31, 33, 35, 39, 45, 48, 52, 58 62, 66, 68, 70, 73 and/or 82, but also of the proto-oncogenic types HPV 5, 8, 14, 17, 20 and/or 47 or of papilloma relevant types HPV 6, 11, 13, 26, 28, 32 and/or 60.

The lipoprotein envelope of the VLP may include a fusion protein. The terms “fusion protein” or “fusogenic glycoprotein” herein refer to viral type I transmembrane proteins that have been classified into three classes. Class I viral fusion proteins are trimers with a large globular head region and a long a-helical coiled-coil stalk region. Examples of class I viral fusion proteins include, but are not limited to Influenza HA, respiratory syncytial and virus F, HIV gp4. Class II fusion proteins are trimers composed essentially of P-sheets. Examples of class II viral fusion proteins include, but are not limited to, Tick-borne encephalitis virus E, Semliki Forest virus E1, Rift Valley fever virus Gc. Class III viral fusion proteins are five-domain molecules composed of both secondary structure elements: a-helices and P-strands. Examples of class III viral fusion proteins include, but are not limited to vesicular stomatitis virus G, Herpes simplex virus gB or baculovirus gp64. Preferably, the lipoprotein envelope of the VLP used in the composition of the invention include a class III viral fusion protein.

The viral fusion protein or fusogenic protein can be a glycoprotein or a combination of several glycoproteins from retroviridae (including lentivirus and retrovirus, e.g., alpharetrovirus, betaretrovirus, ammaretrovirus, deltaretrovirus, epsilonretrovirus), herpesviridae, poxviridae, hepadnaviridae, flaviviridae, togaviridae, coronaviridae, hepatitis D virus, orthomyxoviridae, paramyxoviridae, filoviridae, rhabdoviridae, bunyaviridae or orthopoxiviridae (e.g., variola). In a preferred aspect, the viral fusogenic glycoprotein is from flaviviridae, retroviridae, orthomyxoviridae, paramyxoviridae, bunyaviridae or hepadnaviridae. In a specific aspect, the viral fusogenic glycoprotein is from orthomyxovirus, rhabdovirus or retrovirus. In a more preferred aspect, the viral fusogenic glycoprotein is a glycoprotein from HIV (Human Immunodeficiency Virus) including HIV-1 and HIV-2, Influenza including Influenza A (e.g., subtypes H5N1 and H1N1) and Influenza B, thogotovirus or VSV (Vesicular Stomatitis Virus).

The virus-like particle preferably further comprises a capsid. Preferably, the capsid is from retroviridae. Retroviridae includes lentivirus and retrovirus, e.g., alpharetrovirus, betaretrovirus, gammaretrovirus, deltaretrovirus and epsilonretrovirus. For instance, the capsid is from human immunodeficiency virus (HIV) including HIV-1 and HIV-2, simian immunodeficiency virus (SIV), feline immunodeficiency virus (FIV), Puma lentivirus, bovine immunodeficiency virus (BIV), Caprine arthritis encephalitis virus, feline leukemia virus (FeLV), murine leukemia virus (MLV), bovine leukemia virus (BLV), human T-lymphotropic virus (HTLV, e.g., HTLV-1,-2,-3 or -4), Rous sarcoma virus (RSV), Avian sarcoma leucosis virus, Equine infections anemia virus, Moloney Murine leukemia virus (MMLV). More preferably, the retroviral capsid is from HIV or MLV. Preferably the capsid is from a lentivirus or a retrovirus.

Optionally, the viral glycoprotein can be fused or covalently bound to an antigen of interest or any other protein or nucleic acid of interest, preferably a tumor associated antigen, or a combination thereof. A non-exhaustive list of antigens which can be further included in VLPs, in addition to the viral glycoprotein and capsid proteins is disclosed hereafter. More specifically, VLPs can also include antigens from tumor associated antigens such as Her2/neu, CEA (carcinoembryogenic antigen), HER2/neu, MAGE2 and MAGE3 (Melanoma-associated antigen), RAS, mesothelin or p53, from HIV such as Vpr, Vpx, Vpu, Vif and Env, from bacteria such as C. albicans SAP2 (secreted aspartyl proteinase 2), Clostridium difficile, from parasites such as Plasmodium falciparum proteins such as CSP (circumsporozoite protein), AMA-1 (apical membrane antigen-1), TRAP/SSP2 (sporozoite surface protein 2, LSA (liver stage antigen), Pf Exp1 (Pf exported protein 1), SALSA (Pf antigen 2 sporozoite and liver stage antigen), STARP (sporozoite threonine and asparagines-rich protein) or any protein as disclosed in international patent application WO2011/138251.

Composition of VLPs and methods for producing them are disclosed in details in EP 3430147 B1.

As already indicated, in a particular aspect herein described, the STING activator is a small molecule.

The terms “small molecule” herein refer to any compounds, of natural or synthetic origin, that targets and binds to STING and activates the cGAS-STING pathway, thereby promoting IKK-related kinase TANK-binding kinase 1 (TBK1) signaling and activating nuclear factor-kappa B (NF-kB) and interferon regulatory factor 3 (IRF3) in immune cells in the tumor microenvironment (TME). This leads to the production of pro-inflammatory cytokines, including interferons (IFNs), and more specifically to the production/expression of IFN-beta (IFN-β). The binding of small molecules to STING results in a cytotoxic T-lymphocytes (CTLs)-mediated immune response against tumor cells and causes tumor cell lysis. Because of their discrepancies in chemical structure, and for the purpose of the present invention, the small molecules encompass CDNs, including both natural and synthetic CDNs, and STING agonists that are CDNs-unrelated agents such as flavonoids and xanthone derivatives as well as diaminobenzimidazoles (diABZIs).

Among flavonoids, flavone acetic acid (FAA) is the first identified STING agonist. In an attempt to improve efficacy, various modifications were introduced into the molecular structure of FAA, resulting in a battery of derivatives including 5,6-dimethylxanthenone-4-acetic acid (DMXAA, also known as ASA404 or vadimezan).

α-mangostin is a xanthone derivative with allyl and hydroxyl groups substituted in the xanthone scaffold. Despite a weaker potency for inducing type I IFN compared with cGAMP in reporter assays, α-mangostin is an interesting STING agonist usable in the context of the herein described therapeutic uses.

FAA and dimethyloxoxanthenyl acetic acid (DMXAA) are specific mouse (m)STING agonists and α-mangostin shows higher affinity to human (h)STING than to mSTING.

Diaminobenzimidazole (also named diABZIs or amidobenzimidazole dimers) exhibits enhanced binding to STING and cellular function as compared to cGAMP. Moreover, diABZIs triggered a STING-dependent activation of type I IFN and proinflammatory cytokines. Such compounds, together with bispyrazole di acid or bis-phenol dimer, are disclosed in international patent applications WO 2017/175147 and WO 2019/069270.

E7766 belongs to a novel class of macrocycle-bridged STING agonists (MBSAs). E7766, a MBSA derivative of ADU S100, is the only STING agonist currently being tested in cancer patients as a standalone intravenous intervention. Tolerability, safety and preliminary activity of this molecule have been investigated in patients with advanced solid tumors or lymphomas (NCT04144140) as well as in individuals affected by non-muscle invasive bladder cancer (NCT04109092).

Other STING agonist small molecules are currently under development, such as for example MK-2118, GSK3745417, IMSA-101, TAK-676, CDR5500, TTI-10001 and SEL312-4787.

Preferably, the STING activator is a small molecule selected from a dithio-(RP, RP)-[cyclic[A(2′,5′)pA(3′,5′)p]] (ML RR-S2 CDA, also named ADU-S100); MK-1454; 5,6-dimethyl-xanthenone acetic acid (DMXAA); Flavone-8-acetic acid (FAA); 10-carboxymethyl-9-acridanone (CMA); α-Mangostin (xanthone); ML RR-S2 CDG; ML RR-S2 cGAMP; 6-bromo-N-(naphthalen-1-yl)benzo[d][1,3]dioxole-5-carboxamide (BNBC); dispiro diketopiperzine (DSDP); 2′3′-cG^{S}A^{S}MP (a bis-phosphothioate analog of 2′3′-cGAMP); a macrocycle-bridged STING agonist (MBSA); E7766 (a MBSA derivative of ADU-S100); a benzothiophene oxobutanoic acid (MSA-2) derivative; SB 11285; diamidobenzimidazole (diABZI); IMSA-101, TAK-676; CRD5500; TTI-10001; and SEL312-4787.

By the expression “a nucleic acid coding for a STING activator” is meant a polynucleotide which directly specifies the amino acid sequence of a polypeptide. The boundaries of the coding sequence are generally determined by an open reading frame which begins with a start codon such as ATG, GTG, or TTG and ends with a stop codon such as TAA, TAG, or TGA. The coding sequence may be a genomic DNA, cDNA, synthetic DNA, or a combination thereof.

The above-mentioned nucleic acid coding for a STING agonist can be packed into a vector that optimizes its transcription and translation. The term “vector” means a linear or circular DNA molecule that comprises a polynucleotide encoding the STING agonist of the invention and is operably linked to control sequences allowing its expression. The expression vector comprises an expression cassette suitable for expressing the STING agonist of the invention. Expression vectors that can be used in the present invention include non-exhaustively eukaryotic expression vectors, in particular mammalian expression vectors, virus-based expression vectors, baculovirus expression vectors, plant expression vectors, and plasmid expression vectors. Suitable expression vector can be derived from viruses such as baculoviruses, papovaviruses such as for example SV40, vaccinia viruses, adenoviruses, fowl pox viruses, pseudorabies viruses or retroviruses, especially from lentiviruses.

The vector can also be of viral origin. For example, an adenovirus that produces the bacterial STING agonist c-di-GMP. Alternatively, the coding sequence of the STING agonist can be incorporated in a cell, either in the cell's chromosome or in its cytoplasm. Any eukaryotic cell can be used in the method. For instance, cells used for the production can be mammalian cells, for example COS-1 cells, CHO (Chinese hamster ovary) cells (U.S. Pat. Nos. 4,889,803; 5,047,335), HEK (human embryonic kidney) cells, cells from cell lines such as 293, 293T, HL-116, Vero and BHK (baby hamster kidney) cell lines; a plant cell (e.g., N. bethamiana); an insect cell such as Spodopterafrugiperda (5/J-derived cells such as Sf-9 cells), SF21, Hi-5, Express Sf+, and S2 Schneider cell, in particular with baculovirus-insect cell expression system; or an avian cell.

In another aspect, the STING activator can be an antibody conjugated with a STING agonist, such as for example SB 11325/11396 for which the specific target remains unknown at the date of the present invention. The terms “immunoconjugate” and “antibody conjugate” are used interchangeably herein. Antibody conjugates comprising agonists of STING are disclosed in international patent application WO2018/200812.

Hydrolysis of 2′3′-cGAMP by phosphodiesterase can be a barrier to the activation of the STING signaling pathway. Recently, several inhibitors of ectonucleotide pyrophosphatase phosphodiesterase 1 (ENPP1) have been designed and developed to activate STING indirectly, such as MAVU-104, MV626 and SR8314.

In another aspect, the STING activator can be an inhibitor of ectonucleotide pyrophosphatase phosphodiesterase 1 (ENPP1) such as MAVU-104.

The composition/combination of the invention also comprises an (i.e., possibly several) anti-regulatory T cells (Tregs) agent(s), preferably an anti-intratumoral Tregs agent, as herein disclosed.

By the expression “regulatory T cells” is meant subpopulations of T cells, in particular subpopulations of T cells located in the tumor, that express markers such as IL2ra (CD25), Ikzf2 (Helios), Ikzf4 (Eos), IL2rb (CD122), Socs2, Nrpl, Ebi3 or CTLA. Immunosuppressive activity of Treg cells refers, for the skilled person in the art, to the ability of Treg cells to suppress proliferation of CD25-CD4⁺ T and CD8⁺ T cells. T cells proliferation assay is the gold standard for assessing Treg immunosuppressive activity. Multiple subpopulations of Tregs, constitutive and inducible, CD4⁺ and CD8⁺, FoxP3⁺ and FoxP3-, have been described in the context of malignancy. Most studies associate the presence of CD4⁺CD25⁺FoxP3⁺ Tregs in tumors with poor prognosis.

The terms “Treg” or “regulatory T cell” refer to CD4⁺ T cells that suppresses CD4⁺CD25- and CD8⁺ T cell proliferation and/or effector function, or that otherwise down-modulate an immune response. Notably, Treg may down-regulate immune responses mediated by Natural Killer (NK) cells, Natural Killer T cells as well as other immune cells. In a preferred aspect, Tregs of the invention are Foxp3*.

The terms “regulatory T cell function” or “a function of Treg” are used interchangeably to refer to any biological function of a Treg that results in a reduction in CD4⁺CD25- or CD8⁺T cell proliferation or a reduction in an effector T cell-mediated immune response. Treg function can be measured via techniques established in the art. Non-limiting examples of useful in vitro assays for measuring Treg function include Transwell suppression assay as well as, more generally, in vitro assays in which the target conventional T cells (Tconv) and Tregs purified from human peripheral blood or umbilical cord blood (or murine spleens or lymph nodes) are optionally activated by anti-CD3⁺ anti-CD28 coated beads (or antigen-presenting cells (APCs) such as, e.g., irradiated splenocytes or purified dendritic cells (DCs) or irradiated PBMCs) followed by in vitro detection of conventional T cell proliferation (e.g., by measuring incorporation of radioactive nucleotides (such as, e.g., [3H]-thymidine) or fluorescent nucleotides, or by Cayman Chemical MTT Cell Proliferation Assay Kit, or by monitoring the dilution of a green fluorochrome ester CFSE or Seminaphtharhodafluor (SNARF-1) dye by flow cytometry). Other common assays measure T cell cytokine responses. Useful in vivo assays of Treg function include assays in animal models of diseases in which Tregs play an important role, including, e.g., (1) homeostasis model (using naive homeostatically expanding CD4⁺ T cells as target cells that are primarily suppressed by Tregs), (2) inflammatory bowel disease (IBD) recovery model (using Th1 T cells (Th17) as target cells that are primarily suppressed by Tregs), (3) experimental autoimmune encephalomyelitis (EAE) model (using Th17 and Th1 T cells as target cells that are primarily suppressed by Tregs), (4) B16 melanoma model (suppression of antitumor immunity) (using CD8⁺ T cells as target cells that are primarily suppressed by Tregs), (5) suppression of colon inflammation in adoptive transfer colitis where naive CD4⁺CD45⁺RBhi Tconv cells are transferred into Rag1^{-/-} mice, and (6) Foxp3 rescue model (using lymphocytes as target cells that are primarily suppressed by Tregs). According to one protocol, all of the models require mice for donor T cell populations as well as Rag1^{-/-} or Foxp3 mice for recipients. For more details on various useful assays see, e.g., Collison and Vignali, In Vitro Treg Suppression Assays, Chapter 2 in Regulatory T Cells: Methods and Protocols, Methods in Molecular Biology, Kassiotis and Liston eds., Springer, 2011, 707:21-37; Workman et al., In Vivo Treg Suppression Assays, Chapter 9 in Regulatory T Cells: Methods and Protocols, Methods in Molecular Biology, Kassiotis and Liston eds., Springer, 2011, 119-156; Takahashi et al., Int. Immunol., 1998, 10:1969-1980; Thornton et al., J. Exp. Med., 1998, 188:287-296; Collison et al., J. Immunol., 2009, 182:6121-6128; Thornton and Shevach, J. Exp. Med., 1998, 188:287-296; Asseman et al., J. Exp. Med., 1999, 190:995-1004; Dieckmann et al., J. Exp. Med., 2001, 193:1303-1310; Belkaid, Nature Reviews, 2007, 7:875-888; Tang and Bluestone, Nature Immunology, 2008, 9:239-244; Bettini and Vignali, Curr. Opin. Immunol., 2009, 21:612-618; Dannull et al., J Clin Invest, 2005, 115(12):3623-33; Tsaknaridis et al., J Neurosci Res., 2003, 74:296-308.

By the expression “anti-regulatory T cells (Tregs) agent” or “anti-intratumoral regulatory T cells (Tregs) agent” is meant a compound able to modulate the different subpopulations of Tregs, in particular Tregs subpopulations present in the tumor, either by reducing the number of Tregs or by modulating their phenotypic signature. In a preferred aspect, the anti-regulatory T cells (Tregs) agent used in the composition/combination of the invention particularly target CD45⁺ TCRb⁺CD4⁺ Tregs, in particular CD25⁺FoxP3⁺ and FoxP3⁺Ki67⁺ Tregs subpopulations, in particular Tregs subpopulations located in the tumor.

In a particular aspect herein described, the composition of the invention does not comprise a purinergic receptor agonist. In particular, the anti-regulatory T cells (Tregs) agent, preferably the anti-intratumoral Tregs agent, is not a purinergic receptor agonist.

In a particular aspect herein described, the anti-regulatory T cells (Tregs) agent, preferably the anti-intratumoral Tregs agent, is selected from imatinib or a derivative or salt thereof, dasatinib or a derivative or salt thereof, an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof. For example, the anti-regulatory T cells (Tregs) agent, preferably the anti-intratumoral Tregs agent, is selected from imatinib or a derivative or salt thereof, an anti-CTLA-4 antibody having an IgG2 constant region or a mutated IgG1 constant region; an anti-CD25 antibody; and any combination thereof.

CTLA4, LAG3, TIM-3 and ICOS are immune checkpoints. Agonists of these immune checkpoints, in particular antibodies, act as immune checkpoint inhibitors. By the terms “immune checkpoint inhibitor” is meant any drug that blocks proteins called checkpoints that are made by some types of immune system cells, such as T cells, and some cancer cells. These checkpoints help keeping immune responses from being too strong and sometimes can keep T cells from killing cancer cells. When these checkpoints are blocked, T cells can kill cancer cells more efficiently. Examples of checkpoint proteins found on T cells or cancer cells include, but are not limited to, LAG3/MHC-II and CTLA-4/B7-1/B7-2. In the context of the present invention, the herein described immune checkpoints of interest are those ubiquitous on Tregs.

Preferably, the anti-regulatory T cells (Tregs) agent is selected from an anti-CTLA-4 (also known as CD152) having an IgG2 constant region, such as tremelimumab (CP-675,206), or having a mutated IgG1 constant region such as such as BMS 986249 (or also named CTLA4-Probody), BMS-986288 (or also named CTLA4-NF), Zalifrelimab (or also referred as AGEN1884), Quavonlimab (or also referred as MK-1308), HBM4003 (from Harbour BioMed), ONC-392, or such as herein below described; an anti-LAG3 monoclonal antibody such as Sym-2011, MK-4280, TSR-033, IMP321, Relatlimab (BMS986016), LAG525, REGN3767, MGD013, FS118, INCAGN02385 or EOC202; an anti-TIM-3 monoclonal antibody such as Sym-023, BMS-986258, LY3321367, SHR-1702 or cobolimab (TSR-022); an anti-ICOS monoclonal antibody such as JTX-2011, GSK3359609, BMS-986226, KY1044 or MEDI-570. More preferably, the anti-LAG3, the anti-TIM-3 or the anti-ICOS antibody(ies) has an IgG2 constant region or a mutated IgG1 constant region (as further defined herein below).

CD25, CCR4, CCR8 and TNFR2 are receptors present at the surface of T cells, in particular of Tregs.

Preferably, the anti-regulatory T cells (Tregs) agent is selected from an anti-CD25 antibody such as daclizumab, RG-6292 (R07296682), or camidanlumab tesirine (ADCT-301); an anti-CD25 antibody coupled to a toxin such as RFT5-dgA immunotoxin (IMTOX25); an anti-CCR4 antibody such as mogamulizumab; an anti-CCR8 antibody such as BMS-986340, JTX-1811, SRF114, HBM1022, MAB1429, FPA157; an anti-TNFR2 antibody. More preferably, the anti-CD25, the anti-CCR4, the anti-CCR8 or the anti-TNFR2 antibody(ies) has an IgG2 constant region or a mutated IgG1 constant region (as further defined herein below).

More preferably, the anti-Tregs agent, preferably the anti-intratumoral Tregs agent, is an anti-CTLA-4 antibody of IgG1 isotype that has been engineered, typically mutated in its Fc constant region and/or in a Fab region, preferably at specific herein described positions. The engineering of the CH1, CH2 and/or CH3 constant regions corresponds to an intentional human manipulation of the genetic sequence. The mutation is preferably a substitution of an amino acid by another one. The mutations defined herein below concerning the Fc constant region of an anti-CTLA-4 antibody of IgG1 subtype are also transposable to a Fc constant region of an anti-LAG3, an anti-TIM-3, an anti-ICOS, an anti-CD25, an anti-CCR4, an anti-CCR8 or an anti-TNFR2 antibody.

In a preferred aspect, the anti-intratumoral regulatory T cells (Tregs) agent is an anti-CTLA-4 antibody wherein the mutation of the IgG1 constant region is located in a CH2 domain (of the (Fc) constant region).

In a further preferred aspect, the anti-intratumoral Tregs agent is an anti-CTLA-4 antibody wherein the mutation of the IgG1 constant region is located in a CH3 domain (of the (Fc) constant region).

In another preferred aspect, the anti-intratumoral Tregs agent is an anti-CTLA-4 antibody wherein the mutation of the IgG1 constant region is located in a CH1 domain (of a Fab region).

In a further preferred aspect, the anti-intratumoral regulatory T cells (Tregs) agent is an anti-CTLA-4 antibody comprising at least two mutations located in the CH1, CH2 and/or CH3 domains.

Preferred amino acid substitutions performed in the Fc region, are responsible for the enhancement of an effector function of the anti-CTLA-4 antibody of IgG1 isotype, in particular of the antibody dependent cellular cytotoxicity (ADCC) activity. The Fc constant region may be modified to increase ADCC and/or to increase the affinity for an Fcy receptor (FcyR) by modifying one or more amino acids at the following positions of the CH2 (corresponding to amino acids positions 113-223 of SEQ ID NO: 1) or CH3 domains (corresponding to amino acids positions 224-329 of SEQ ID NO: 1): 233, 234, 235, 236, 237, 238, 239, 243, 247, 256, 262, 267, 268, 270, 271, 280, 290, 292, 298, 300, 305, 324, 326, 328, 330, 332, 333, 334, 339 or 396, this last numbering being that of the EU index or equivalent in the Kabat scheme. The numbering of the amino acid positions results from the IGHG1 amino acid translation of the sequence J00228 (SEQ ID NO: 1), now replaced in databases by sequence AH007035. J00228 corresponds to the IGHG1*01 allele (Alignment of alleles: Human IGHG1) and to a G1 ml,17 chain (G1m allotypes). The EU gamma 1 chain is encoded by the IGHG1*03 allele (CH1 K120>R, CH3 D12>E and L14>M) and is a Glm3 chain (G1m allotypes). EU numbering was defined by Edelman et al., (Proc. Natl. Acad. USA, 63, 78-85 (1969)). Kabat numbering was disclosed in Kabat et al., Sequences of proteins of immunological interest. 5th Edition —US Department of Health and Human Services, NIH publication n°91-3242, pp 662,680,689 (1991).

Preferably, the mutation of the IgG1 occurs in the CH2 and/or CH3 domain.

In one aspect, the first CH2 domain of the Fc constant region of the anti-CTLA4 antibody comprises at least one mutation at an amino acid position selected from 234, 235, 236, 239, 268, 270, 298, preferably L234Y/L235Q/G236W/S239M/H268D/D270E/S298A substitutions, and the second CH2 domain of the Fc constant region of the anti-CTLA4 antibody comprises at least one mutation at an amino acid position selected from 270, 326, 330 and 334, preferably D270E/K326D/A330M/K334E substitutions.

In another aspect, each of the two CH2 domains of the Fc constant region of the anti-CTLA4 antibody comprises at least one mutation at an amino acid position selected from 239, 330 and 332, preferably S239D/A330L/I332E substitutions.

In another aspect, at least one CH2 domain of the Fc constant region of the anti-CTLA4 antibody comprises mutation(s) at amino acid position(s) 236, 238, 239, 267 and/or 328, preferably substitution(s) selected from G236D, P238D, S239D, S267E, L328F, L328E and any combination thereof.

In another aspect, at least one CH2 domain of the Fc constant region of the anti-CTLA4 antibody comprises the substitution P238D and one or more substitutions selected from the group consisting of:
E233D, G237D, H268D, P271G, and A330R;
V262E, S267E, and L328F; and
V264E, S267E and L328F.

In another aspect, at least one CH2 domain of the Fc constant region of the anti-CTLA4 antibody comprises at least one mutation at an amino acid position selected from 236, 239, 267, 268, 324, 332, preferably at least one substitution selected from 236A, 239D, 239E, 267E, 268D, 268E, 268F, 324T, 332D and 332E.

In another aspect, at least one CH2 and/or one CH3 domain of the Fc constant region of the anti-CTLA4 antibody comprises at least one mutation at an amino acid position selected from 236, 239, 243, 256, 290, 292, 298, 300, 305, 330, 332, 333, 334, 339, 396, preferably at least one substitution selected from G236A, S239D, F243L, T256A, K290A, R292P, S298A, Y300L, V305I, A330L, 1332E, E333A, K334A, A339T and P396L.

In another aspect, at least one CH2 and/or one CH3 domain of the Fc constant region of the anti-CTLA4 antibody comprises at least one mutation at an amino acid position selected from 243, 247, 280, 290, 292, 298, 300, 305, 326, 333, 334, 339, 396, preferably at least one substitution selected from 243L, 2471, 280H, 290S, 292P, 298A, 298D, 298V, 300L, 305I, 326A, 333A, 334A, 339D, 339Q and 396L.

Still preferably, the mutation of the IgG1 occurs in the CH1 domain (of a Fab region) (corresponding to amino acids positions 1-97 of SEQ ID NO: 1) and/or CH2 domain (of the Fc region).

In one aspect, at least one CH1 and/or one CH2 domain region of the anti-CTLA4 antibody comprises at least one mutation at an amino acid position selected from 135, 137, 139, 181, 216, 217, preferably at least one substitution selected from M135Y, S137T, T139E, S181A, E216A and K217A.

Another suitable anti-CTLA-4 antibody usable in a composition of the invention is tremelimumab (CP-675,206). Tremelimumab is an IgG2 antibody that binds well to FCGRIIA but not FCGRIIIA.

The anti-CTLA-4 antibody used in the composition of the invention does not have wild type (i.e., not mutated/engineered) IgG1 constant region. In particular, the anti CTLA-4 antibody used in the composition of the invention is not ipilimumab.

In a preferred aspect, the anti-CTLA-4 antibody present in the composition of the invention is an IgG2, in particular an IgG2A (isotype 2A) anti-CTLA-4 antibody.

Preferably, the anti-CTLA-4 antibody, or the fragment thereof, has a binding affinity for CTLA-4 of about 10⁻⁸M, preferably about 10⁻⁹M, more preferably about 10⁻⁰M, even more preferably 10⁻¹¹M.

In a particular aspect herein described, the anti-regulatory T cells (Tregs) agent, preferably the anti-intratumoral Tregs agent, is imatinib or a derivative or salt thereof.

The term “imatinib” designates a compound (CAS number: 152459-95-5) that binds to a specific enzyme, called Bcr-Abl tyrosine kinase, responsible for the increased production of white blood cells. It also inhibits other several TK receptors: Kit, the SCF (Stem Cell Factor) receptor encoded by the proto-oncogene c-Kit, the discoidin domain receptors (DDR1 and DDR2), the CSF-1R (Colony Stimulating Factor Receptor) and the PDGF (Platelet-Derived Growth Factor: PDGFR-alpha and PDGFR-beta) receptors. Imatinib may also inhibit cellular processes mediated by activation of these receptor kinases. Commercially available imatinib is GLIVEC™

In another particular aspect, the anti-regulatory T cells (Tregs) agent, preferably the anti-intratumoral Tregs agent, is dasatinib or a derivative or salt thereof.

The term “dasatinib” designates a dual BCR/ABL and Src family tyrosine kinase inhibitor (CAS number: 302962-49-8). The main targets of dasatinib, are Breakpoint Cluster RegionAbelson (BCRABL), SRC, Ephrins and Growth Factor (GFR). Commercially available dasatinib is SPRYCEL™

In another particular aspect, a combination of distinct antiregulatory Tregs agents is used in the composition of the invention.

In another particular aspect, the STING activator is a VLP. This VLP may be used together with an anti-CTLA4 antibody having an IgG2 constant region or a mutated IgG1 constant region and with another distinct anti-Tregs agent, in particular with another distinct immune checkpoint inhibitor, as herein described.

The present invention also relates to a pharmaceutical composition/combination as herein described, in particular a therapeutic, vaccine or veterinary composition, for use as a medicament.

The composition/combination of the invention can be used as a drug/medicament, for example as a vaccine or a vaccine adjuvant. Hence, the present invention also relates to a pharmaceutical composition/combination, for example a therapeutic, vaccine or veterinary composition, comprising i) a STING (stimulator of interferon genes) activator as herein described, preferably in a vectorized form, i.e., a vector comprising (containing or expressing) a STING activator, and ii) an anti-regulatory T cells (Tregs) agent, preferably an anti-intratumoral Tregs agent, as herein described.

In a particular aspect, the present invention also relates to a pharmaceutical composition/combination, for example a therapeutic, vaccine or veterinary composition, comprising i) a STING (stimulator of interferon genes) activator as herein described, preferably in a vectorized form, i.e., a vector comprising (containing or expressing) a STING activator, and ii) an anti-intratumoral regulatory T cells (Tregs) agent as herein described.

The composition may further comprise an adjuvant. The composition may also comprise or be administered in combination with one or more additional therapeutically active substances. Pharmaceutical, vaccine and veterinary compositions comprising i) a STING (stimulator of interferon genes) activator, preferably in a vectorized form, i.e., a vector comprising (containing or expressing) a STING activator, ii) an anti-regulatory T cells (Tregs) agent, preferably an anti-intratumoral regulatory T cells (Tregs) agent and a pharmaceutically acceptable carrier or excipient are in particular herein described.

Pharmaceutical compositions/combinations of the present invention may indeed comprise a pharmaceutically acceptable carrier, support or excipient, which, as used herein, may be or may comprise solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams and Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. Pharmaceutically acceptable excipients can be preservative, inert diluent, dispersing agent, surface active agent and/or emulsifier, buffering agent and the like. Suitable excipients include, for example, water, saline, dextrose, sucrose, trehalose, glycerol, ethanol, or similar, and combinations thereof. In addition, if desired, the vaccine may contain auxiliary substances such as wetting or emulsifying agents, pH buffering agents, or adjuvants which enhance the effectiveness of the composition. In some aspects, herein described (pharmaceutical) compositions comprise one or more preservatives. In some aspects, herein described (pharmaceutical) compositions comprise no preservative.

The (pharmaceutical) compositions/combinations as disclosed herein may comprise an adjuvant. Any adjuvant may be used in accordance with the present invention. A large number of adjuvants are known. A useful compendium of many such compounds has been prepared by the National Institutes of Health and can be found by the skilled person in the art (www.niaid.nih.gov/daids/vaccine/pdf/compendium.pdf). See also Allison (1998, Dev. Biol. Stand., 92:3-11; incorporated herein by reference), Unkeless et al. (1998, Annu. Rev. Immunol., 6:251-281; incorporated herein by reference), and Phillips et al. (1992, Vaccine, 10: 151-158; incorporated herein by reference). Hundreds of different adjuvants are known in the art and may be employed in the practice of the present invention. Exemplary adjuvants that can be utilized in with the context of the invention include, but are not limited to, cytokines, gel-type adjuvants (e.g., aluminum hydroxide, aluminum phosphate, calcium phosphate, etc.); microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, saponins, etc.); synthetic adjuvants (e.g., nonionic block copolymers, muramyl peptide analogues, polyphosphazene, synthetic polynucleotides, etc.); and/or combinations thereof. Other exemplary adjuvants include some polymers (e.g., polyphosphazenes described in U.S. Pat. No. 5,500,161), Q57, QS21, squalene, tetrachlorodecaoxide, etc.

The invention relates in particular to a composition/combination as herein described for use in prevention or treatment of cancer or of a STING-mediated disease or disorder in a subject, in particular a STING-mediated cancer.

The pharmaceutical compositions/combinations as disclosed herein are useful for inducing or stimulating/enhancing an immune response/effect in a subject in need thereof. The present invention relates to a method for inducing or enhancing an immune response in a subject in need thereof, comprising a step of decreasing or inhibiting the immunosuppressive activity of Treg cells, preferably of Tregs cells present in the tumor, by administering a therapeutically efficient amount of a pharmaceutical composition as disclosed herein, comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, as herein described to a subject in need thereof, wherein decreasing or inhibiting the activity of the Treg cells, preferably of Tregs cells present in and around (i.e. in the microenvironment of) the tumor, induces in the subject a therapeutic effect against the disease the subject is suffering of, in particular cancer. An immune response may refer to cellular immunity, humoral immunity or may involve both. An immune response may also be limited to a part of the immune system.

For example, in certain aspects, the immunogenic composition herein described may induce an increased IFNy response. In certain aspects, the immunogenic composition herein described may induce a mucosal IgA response (e.g., as measured in nasal and/or rectal washes). In certain aspects, the immunogenic composition herein described may induce a systemic IgG response (e.g., as measured in serum). In certain aspects, the immunogenic composition herein described may induce virus-neutralizing antibodies or a neutralizing antibody response. In certain aspects, the immunogenic composition herein described may induce a CTL response.

Also herein described is a method of decreasing or inhibiting the immunosuppressive functions in a subject. The method includes a step of decreasing or inhibiting the immunosuppressive activity of Treg cells, preferably of Tregs cells present in the tumor, by administering a pharmaceutical composition comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, as herein described to a subject in need thereof wherein decreasing or inhibiting the activity of the Treg cells, preferably of Tregs cells present in and around (i.e. in the microenvironment of) the tumor, decreases or inhibit the immunosuppressive functions in the subject.

Accordingly, the pharmaceutical compositions/combinations of the invention as herein described are useful as vaccine or as vaccine adjuvant. The composition of the invention can be used to treat a variety of diseases in a patient. The disease can be cancerous or non-cancerous. Cancerous diseases can include cancers that generate tumors as well as cancers that do not produce tumors such as hematological malignancies.

In one aspect, the STING activator and the anti-regulatory T cells (Tregs) agent are co-administered. In another aspect, the STING activator agent(s) and the anti-regulatory T cells (Tregs) agent(s) are co-administered sequentially or concomitantly.

In a preferred aspect, the STING activator and the anti—intratumoral regulatory T cells (Tregs) agent are co-administered. In another preferred aspect, the STING activator agent(s) and the anti—intratumoral regulatory T cells (Tregs) agent(s) are co-administered sequentially or concomitantly.

In one aspect, the present invention relates to a method for treating cancer or a STING-mediated disease or disorder, preferably cancer, or for preventing cancer or a STING-mediated disease or disorder, in particular for preventing cancer relapse, in a subject in need thereof. The method comprises a step of administering a therapeutically efficient amount of a herein described pharmaceutical composition/combination, in particular a pharmaceutical, vaccine or veterinary composition, to a subject in need thereof. The present invention relates in particular to the use of a composition/combination, in particular a pharmaceutical, vaccine or veterinary composition, as disclosed herein for the manufacture of a medicament, for example of a vaccine, for treating cancer in a subject. The present invention also relates to a pharmaceutical composition/combination as disclosed herein for use for treating cancer or preventing cancer relapse.

By “method for treating” is meant a process that is intended to produce a beneficial change in the condition of an individual, e.g., mammal, especially human. Human and veterinary treatments are both contemplated. A beneficial change can include one or more of: restoration of function, reduction of symptoms, limitation or retardation of a disease, disorder, or condition, or prevention, limitation or retardation of deterioration of a patient's condition, disease or disorder, in particular, as used herein, the term “treatment” (also “treat” or “treating”) refers to any administration of an immunogenic composition that partially or completely alleviates, ameliorates, relieves, inhibits, delays onset of, reduces severity of and/or reduces incidence of one or more symptoms or features of a particular disease, disorder, and/or condition or the predisposition toward the disease. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain aspects, the term “treating” refers to the vaccination of a patient.

The term “cancer” encompasses disease or disorder such as cancer, pre-cancerous syndromes and tumor metastasis. The cancer may be a solid or a liquid cancer. Preferably the cancer is a solid cancer. Examples of cancer diseases and conditions in which a composition of the invention may have beneficial antitumor effects include, but are not limited to, cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer; cancer of the anal region; carcinomas of the fallopian tubes, endometrium, cervix, vagina, vulva, renal pelvis, renal cell; sarcoma of soft tissue; myxoma; rhabdomyoma; fibroma; lipoma; teratoma; cholangiocarcinoma; hepatoblastoma; angiosarcoma; hemangioma; hepatoma; fibrosarcoma; chondrosarcoma; myeloma; chronic or acute leukemia; lymphocytic lymphomas; primary CNS lymphoma; neoplasms of the CNS; spinal axis tumours; squamous cell carcinomas; synovial sarcoma; malignant pleural mesotheliomas; brain stem glioma; pituitary adenoma; bronchial adenoma; chondromatous hamartoma; mesothelioma; Hodgkin's Disease or a combination of one or more of the foregoing cancers.

When used for treating solid cancer or preventing solid cancer relapse, the pharmaceutical composition/combination as disclosed herein advantageously depletes the Treg cells present in the tumor and its microenvironment.

The pharmaceutical compositions/combinations as disclosed herein may further be used in combination with one or more second therapeutic agents, in particular chemotherapeutic agent(s) (i.e., cancer treating agent(s)). Chemotherapeutic agents can include, but are not limited to, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, taxol, temozolomide, teniposide, testosterone, thioguanine, thiotepa, titanocene dichloride, topotecan, trastuzumab, tretinoin, trimetrexate, vinblastine, vincristine, vindesine, vinorelbine, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR) and methotrexate (MTX).

The patient exposed to a pharmaceutical composition as disclosed herein may in addition be exposed to radiotherapy, and/or treated by surgery, hormonotherapy or bone marrow transplantation depending for example on the type of tumor, on the patient condition, and on other health issues.

As used herein, the term “vaccination” refers to the administration of a composition intended to generate an immune response, for example to a disease-causing agent (e.g., a virus). For the purposes of the present invention, vaccination can be administered before, during and/or after exposure to a disease-causing agent, and in certain aspects, before, during, and/or shortly after exposure to the agent. In some aspects, vaccination includes multiple administrations, appropriately spaced in time, of a vaccinating composition. As used herein, the term “therapeutically effective amount” refers to an amount (or dose) sufficient to confer a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a composition of the invention effective to prevent, ameliorate or treat a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular immunogenic composition, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent(s) employed; the specific composition employed; the age, body weight, general health, sex and diet of the patient; the time of administration, route of administration, and/or rate of excretion or metabolism of the specific immunogenic composition employed; the duration of the treatment; and like factors as is well known in the medical arts. The amount of a given compound that will correspond to such an amount will vary depending upon factors such as the particular compound [e.g., the potency (pIC50), efficacy (EC50), and the biological half-life of the particular compound], disease condition and its severity, the identity (e.g., age, size and weight) of the patient in need of treatment, but can nevertheless be routinely determined by one of ordinary skill in the art. Likewise, the duration of treatment and the time period of administration (time period between dosages and the timing of the dosages, e.g., before/with/after meals) of the compound will vary according to the identity of the subject in need of treatment (e.g., weight), the particular compound and its properties (e.g., pharmacokinetic properties), disease or disorder and its severity and the specific composition and method being used, but can nevertheless be determined by one of ordinary skill in the art.

The term “dose” is used herein below in relation with a particular constituent present in a “unit” dose of the composition of the invention, i.e., as explained herein above, in a dose which may be administered to a subject in need of treatment, typically in a subject suffering of cancer or of a STING-mediated disease or disorder, in particular of a cancer, or in whom cancer or a STING-mediated disease or disorder, in particular cancer, should be prevented. As explained above, the terms “dose” and “amount” are equivalent.

In a particular aspect, the dose of the cyclic dinucleotides (preferably packaged into virus-like particles), for example cGAMP, present in the composition of the invention ranges from about 1 ng to about 100 mg, and the dose of imatinib present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-CTLA-4 antibody present in the composition of the invention ranges for about 10 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the dasatinib present in the composition of the invention ranges for about 10 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-LAG3 antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-TIM-3 antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-ICOS antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-CD25 antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-CCR4 antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-CCR8 antibody present in the composition of the invention ranges for about 1 mg to about 1 g.

In another particular aspect, the dose of the cyclic dinucleotides (preferably packaged into virus-like particles), for example cGAMP, present in the composition of the invention ranges from about 1 ng to about 100 mg, and the dose of the anti-TNFR2 antibody present in the composition of the invention ranges for about 1 mg to about 1 g.

As used herein, the term “amelioration” or “improvement” is meant the prevention, reduction or palliation of a state, or improvement of the state of a subject. Amelioration includes, but does not require complete recovery or complete prevention of a disease, disorder or condition. The term “prevention” refers to a delay of onset of a disease, disorder or condition. Prevention may be considered complete when onset of a disease, disorder or condition has been delayed for a predefined period of time. As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment. “dosing regimen” (or “therapeutic regimen”), as that term is used herein, is a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which are separated from one another by a time period of the same length; in some aspects, a dosing regimen comprises a plurality of doses and at least two different time periods separating individual doses.

As used herein, the terms “subject,” “individual” or “patient” refer to a mammal, in particular to human or a non-human mammalian subject whatever its age or sex. The individual (also referred to as “patient” or “subject”) being treated is an individual (fetus, infant, child, adolescent, or adult) suffering from a cancer. In some aspects, the subject is a human. In some other aspects herein described, the subject is an animal, especially a pet (e.g., cat and dog), a farm animal (e.g., cattle, pig, sheep, rabbit, swine, fish, poultry), or a horse.

The pharmaceutical composition/combination of the invention can be in a particular aspect administered, or is suitable for administration, by any systemic or local route of administration. For instance, the route can be a parenteral route such as intraperitoneal, subcutaneous, intramuscular, intravenous, intradermal, intrathecal, intra-arterial, intra-articular and intramedullary route; or an enteral route such as oral and mucosal (e.g., sublingual, intranasal, intra-rectal, intra-vaginal, or intrabronchial) routes. The preferred administration routes are intraperitoneal, subcutaneous, intravenous and oral routes.

For example, pharmaceutical compositions provided here may be provided in a sterile injectable form (e.g., a form that is suitable for subcutaneous injection or intravenous infusion). For example, in some aspects, pharmaceutical compositions are provided in a liquid dosage form that is suitable for injection/infusion. In some aspects, pharmaceutical compositions are provided as powders (e.g., lyophilized and/or sterilized), optionally under vacuum, which are reconstituted with an aqueous diluent (e.g., water, buffer, salt solution, etc.) prior to injection. In some aspects, pharmaceutical compositions are diluted and/or reconstituted in water, sodium chloride solution, sodium acetate solution, benzyl alcohol solution, phosphate buffered saline, etc. In some aspects, powder should be mixed gently with the aqueous diluent (e.g., not shaken).

In a particular aspect, the STING activator is to be administered to the subject by systemic route, for example by subcutaneous, oral, intraperitoneal, intramuscular, intradermal or intravenous route, preferably by subcutaneous route.

In a particular aspect, when the STING activator is a virus-like particle (VLP), the VLP is to be administered to the subject by systemic route, for example by subcutaneous, oral, intraperitoneal or intravenous route, preferably by subcutaneous route.

In a particular aspect, the anti-Tregs agent is to be administered to the subject by systemic route, for example by intraperitoneal, oral, subcutaneous or intravenous route, preferably by intraperitoneal route.

Pharmaceutical compositions and as well as parts of the herein described kits can be provided in a form that can be refrigerated and/or frozen. Alternatively, they can be provided in a form that cannot be refrigerated and/or frozen. Optionally, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer).

Formulations of the pharmaceutical compositions and parts of kits described herein may be prepared by any method known or hereafter developed in the art of pharmacology. Such preparatory methods include the step of bringing active ingredients into association with one or more excipients and/or one or more other accessory ingredients, and then, if necessary and/or desirable, shaping and/or packaging the product(s) into a desired single- or multi-dose unit. A pharmaceutical composition in accordance with the invention may be prepared, packaged, and/or sold in bulk, as a single unit dose, and/or as a plurality of single unit doses.

Relative amounts of active ingredients, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1 percent and 100 percent (w/w) of active ingredients.

Compositions described herein will generally be administered in such amounts and for such a time as is necessary or sufficient to induce an immune response. Dosing regimens may consist of a single dose or a plurality of doses over a period of time. The exact amount of an immunogenic composition [e.g., combination of i) a stimulator of interferon genes (STING) activator and ii) an anti-regulatory T cells (Tregs) agent, preferably an anti-intratumoral Tregs agent] to be administered may vary from subject to subject and may depend on several factors. Thus, it will be appreciated that, in general, the precise dose used will be as determined by the prescribing physician and will depend not only on the weight of the subject and the route of administration, but also on the age of the subject and the severity of the symptoms and/or the risk of infection. In a first aspect, a particular amount of the pharmaceutical composition is administered as a single dose. Alternatively, a particular amount of the pharmaceutical composition is administered as more than one dose (e.g., 1-3 doses that are separated by 1-12 months). Instead, a particular amount of the pharmaceutical composition is administered as a single dose on several occasions (e.g., 1-3 doses that are separated by 1-12 months). The pharmaceutical composition may be administered in an initial dose and in at least one booster dose.

The present invention also relates to a kit of part comprising at least two parts, wherein the first part comprises a STING (stimulator of interferon genes) activator as disclosed herein, preferably in a vectorized form, i.e., a vector comprising (containing or expressing) a STING activator, and the second part comprises an anti-regulatory T cells (Tregs) agent, preferably an anti-intratumoral Tregs agent, as herein disclosed, the first and second parts of the kit being preferably in distinct compartments.

In a particular aspect, the first part of the kit comprises a STING (stimulator of interferon genes) activator which is a virus-like particle (VLP) and the second part of the kit comprises an anti-intratumoral regulatory T cells (Tregs) agent which is imatinib or a derivative or salt thereof.

In another particular aspect, the first part of the kit comprises a STING (stimulator of interferon genes) activator which is a virus-like particle (VLP) and the second part of the kit comprises an anti-intratumoral regulatory T cells (Tregs) agent which is dasatinib or a derivative or salt thereof.

In another particular aspect, the first part of the kit comprises a STING (stimulator of interferon genes) activator which is a virus-like particle (VLP) and the second part of the kit comprises an anti-intratumoral regulatory T cells (Tregs) agent which is an anti-CTLA-4 antibody having an IgG2 constant region or a mutated IgG1 constant region as described herein.

In another particular aspect, the first part of the kit comprises a STING (stimulator of interferon genes) activator which is a virus-like particle (VLP) and the second part of the kit comprises an anti-intratumoral regulatory T cells (Tregs) agent which is an anti-CD25 antibody, in particular an anti-CD25 antibody having an IgG2 constant region or a mutated IgG1 constant region, as described herein.

In one aspect, the part of the kit comprising the STING activator, preferably in a vectorized form, is also in a form adapted for oral, intraperitoneal, intravenous, or for subcutaneous route, preferably for subcutaneous route. The oral route of the composition of the invention is expected to elicit an immune response well adapted in the treatment of non-solid tumors or hematological malignancies.

In one aspect, the part of the kit comprising the anti-regulatory T cells agent, preferably the anti-intratumoral Tregs cells agent, is in a form adapted for oral, intraperitoneal, or intravenous route, preferably for intraperitoneal route.

Depending on the indication to be treated, the first part of the kit comprising the STING activator, preferably the vectorized STING activator, and the second part of the kit comprising the anti-regulatory T cells (Tregs) agent, preferably the anti-intratumoral Tregs cells agent, are co-administered. In one aspect, the first part of the kit comprising the STING activator agent, preferably the vectorized STING activator agent, and the second part of the kit comprising the anti-regulatory T cells (Tregs) agent, in particular the anti-intratumoral Tregs cells agent, are co-administered sequentially or concomitantly.

The present invention also relates to a kit of the invention as herein disclosed for use for treating cancer or a STING-mediated disease or disorder, as described hereinabove. In one aspect, “treat”, “treating” or “treatment” in reference to cancer refers to alleviating the cancer, eliminating or reducing one or more symptoms of the cancer, slowing or eliminating the progression of the cancer, and delaying the reoccurrence of the condition in a previously afflicted or diagnosed patient or subject. In another aspect, “treat”, “treating” or “treatment” in reference to infectious disease refers to alleviating pain, heat, viral load, reducing biofilm formation, etc.

Also herein disclosed is the use of a kit of the invention for the manufacture of a medicament for treating cancer or a STING-mediated disease or disorder in a subject, in particular a STING-mediated cancer.

In another aspect, the present invention relates to a method for stimulating a therapeutic immune effect in a living mammalian subject. The method includes decreasing the immunosuppressive activity of Treg cells, preferably of Treg cells present in a tumor, by administering a therapeutic composition comprising i) a STING (stimulator of interferon genes )activator, preferably the vectorized STING activator, and ii) an anti-regulatory T cells (Tregs) agent, preferably an anti-intratumoral Tregs agent, as herein disclosed to a living mammalian subject wherein decreasing and/or inhibiting the activity of the Treg cells induces a therapeutic effect against the disease in the living mammalian subject.

In a further aspect, the present invention relates to a method of inhibiting or decreasing the immunosuppressive functions in a subject. The method includes inhibiting or decreasing the immunosuppressive activity of Treg cells, preferably of Treg cells present in a tumor, by administering a therapeutic composition comprising i) a STING (stimulator of interferon genes) activator, preferably in a vectorized form, i.e., a vector comprising (containing or expressing) a STING activator, and ii) an anti-regulatory T cells (Tregs) agent, preferably an anti-intratumoral Tregs agent, as herein disclosed to a subject in need thereof wherein decreasing or inhibiting the activity of the Treg cells, preferably in the TME, i.e. in and around the tumor, reduces or inhibits the immunosuppressive functions in the subject.

“Suppression of immune tolerance” as referred to herein relates to suppressing the ability of the subject's immune system to tolerate the presence of disease's antigens including any natural tolerance and/or suppression of tumor avoidance by the subject's immune system.

The methods of the present invention can include a step of administering a composition of the invention to a subject in need thereof wherein administration of the composition can suppress the tolerance of disease and/or disease's antigens by the subject's immune system. Administration of a herein described composition to a subject allows to deactivate the tumor avoidance mechanisms in a subject and leads to better tumor eradication. Preferably, administration of the composition suppresses the activity of Treg cells that are CD4⁺CD25⁺FoxP3⁺. “Treg” cells as referred to herein relate to regulatory T-cells that are CD4⁺CD25⁺FoxP3⁺ and include both nTreg and iTreg. Naive T-cells as referred to herein are T-cells that are CD4⁺CD25⁻FoxP3⁻.

The methods of the present invention include suppressing the activity of Treg cells, in particular Treg cells present in a tumor, in a variety of ways. The suppression of the activity of Treg cells can be, for example, by inhibiting the conversion of naive T-cells to iTreg cells. The method can include a step of administering a composition as described herein that interfere with the conversion of naive cells to Treg cells, for example, by modulating the activity of FoxP3. FoxP3 is a transcription factor that is a marker for cells which are capable of causing immune suppression activity. The absence or reversal of FoxP3 in a cell is an indication that the cell does not, or does no longer, perform suppressive functions.

The methods disclosed herein may be used for veterinary applications, e.g., canine and feline applications. If desired, the methods herein described may also be used with farm animals, such as ovine, avian, bovine, porcine and equine breeds.

The following examples are provided in order to demonstrate and further illustrate certain preferred aspects of the present invention and are not to be construed as limiting the scope thereof.

### Experimental Part

### Example No. 1

### Materials and Methods

### Cell Lines

293T cells were cultured in Dulbecco's Modified Eagle Medium (DMEM) supplemented with 10% fetal bovine serum (FBS, GIBCO) and 1% penicillin-streptomycin (GIBCO). THP-1 cells were cultured in Roswell ParkMemorial Institute (RPMI) 1640 medium with 10% FBS (GIBCO) and 1% penicillin-streptomycin (GIBCO). The cells used for the in vivo experiments were the murine fibrosarcoma cell lines MCA-OVA originally purchased from ATCC. Tumor cells were grown in monolayer at 37° C. with 5% C02 in RPMI 1640 medium supplemented with 10% fetal calf serum (FCS, Biosera), 1% penicillin-streptomycin and 1 mM 13-Mercaptoethanol (GIBCO). Before reaching confluence, tumor cells were harvested with 0.05% trypsin, washed, and suspended in Hank's Balanced Salt Solution (HBSS, GIBCO) for injection. The cell lines were tested negative for a number of pathogens including Mycoplasma ssp. by PCR.

### Mice

All animals were used according to protocols approved by Animal Committee of Curie Institute and maintained in pathogen-free conditions in a barrier facility. C57BL/6J mice were purchased from Charles River Laboratories. Mice were allowed to acclimate to the housing facility for at least three days. All experiments were initiated using female mice between the ages of 6 and 8 weeks.

cGAMP-VLP Production Before Purification

7.5 million 293T cells are plated in a 150 cm² cell culture flask and incubated overnight. The following day, each flask is transfected with 13 μg of murine cGAS (pVAX1-cGAS), 8.1 pg of HIV-1 GAGPOL (psPAX2), 3.3 μg of VSVG (pVAX1-VSVG-INDIANA2), and 50 μL of PEIpro (Ozyme reference POL115-010), according to the manufacturer's instructions. The transfection mixes were made in Opti-MEM (GIBCO). The morning following transfection, the medium was changed with 52 mL of warm VLP production medium (293T culture medium with added 10 mM HEPES and 50 μg/mL Gentamycin) and the cells were incubated at 37° C. with 5% CO₂ until the following day.

cGAMP-VLP Harvest and Purification on Sucrose Cushion

The cGAMP-VLP containing medium was harvested from the cells, centrifuged for 10 minutes at 200 g at 4° C., and filtered on 0.45 μm. 39 mL of cGAMP-VLP containing medium was gently overlaid on 6 mL of cold 20% sterile filtered endotoxin free sucrose in 6 Ultra-Clear tubes (Beckman Coulter, ref 344058), and centrifuged for 1 hour and 30 minutes at 100′000 g at 4° C. The medium and sucrose was gently aspirated, the pellets were resuspended in cold PBS and transferred to one Ultra-Clear 13.2 mL (Beckman Coulter, ref 344059) and centrifuged again at 100′000 g at 4° C. for 1 hour and 30 minutes. The PBS was gently poured out and the pellet was resuspended in the appropriate amount of cold PBS, typically 320 μL.

### THP-1 Activation and SIGLEC-1 Expression Measure

50′000 THP-1 cells were plated in round bottom 96 well plates in 100 μL of medium, and stimulated with 100 μL of cGAMP-VLPs dilutions and a soluble 2′3′cGAMP dilution. The cells were incubated for 18 to 24 hours and stained with an anti-human SIGLEC-1 (Miltenyi ref 130-098-645), fixed in PFA 1% and acquired using a BD FACS Verse cytometer.

### Tumor Growth

Female C57BL/6J mice were inoculated subcutaneously on the lower right flank with 5×10⁵ MCA-OVA cells in 100 μL of PBS. Mice were monitored for body weight loss, morbidity and mortality daily. Tumors were monitored twice or three times per week. Mice were humanely euthanized if ulceration occurred or when tumor volume reached 2000 mm³. Tumor sizes were measured using a digital caliper and tumor volumes calculated with the formula

(length×width²)/2.

Following tumor implantation, mice were randomized into treatment groups using the Randmice software powered by Stimunity.

### In Vivo Immunotherapy

STING systemic (s.c.) therapy consisted of injecting cGAMP-VLP (50 ng cGAMP) in 50 μL of PBS buffer. S.C. injections were initiated when tumors grew to between 35-50 mm³. A U-100 insulin syringe or equivalent: 0.33 mm (29 G)×12.7 mm (0.5 mL) was filled with the composition and all air bubbles removed. Mice were anesthetized with isoflurane. With the bevel facing the skin, the needle was injected shallowly into the area directly adjacent of the tumor, and the needle was moved underneath the skin until it reached the inside back of the tumor (1 cm). The composition was injected slowly into this area close to the tumor. The needle was then removed delicately to avoid reflux.

In vivo Antibody Treg Depletion

For Treg depletion study, MCA-OVA tumor bearing mice were treated with 10 mg/kg Imatinib chemotherapy agent (Imatinib mesylate-SIGMA) or received PBS on days 6, 7, 8, 9 and 10 by intraperitoneal route (i.p.) after implantation with MCA-OVA melanoma. The Treg depletion was confirmed by FACS using tumors and spleen, 72 hours after the last Imatinib injection.

Ex vivo Stimulation Assay

T cell responses were assessed by IFN-γ ELISPOT 10 days after the first s.c. injection of cGAMP-VLP or PBS. Mice were bled in the retro-orbital sinus and PBMCs were isolated from whole blood by lysing the red blood cells. 2×10⁵ PBMCs were plated per well in RPMI medium containing 1% penicillin-streptomycin and stimulated overnight with media as a negative control, 10 μg/mL p15 peptide (KSPWFTTL, SEQ ID NO: 2), 10 μg/mL OVA 257-264 peptide (OVA-1) (SIINFEKL, SEQ ID NO: 3) or 40 μg/mL OVA 265-280 peptide (OVA-2) (TEWTSSNVMEERKIKV, SEQ ID NO: 4). Spots were developed using a mouse IFN-γ ELISPOT antibody (Diaclone) according to the manufacturer's instructions, and the number of spots enumerated using an ImmunoSpot analyzer.

### LEGENDplex™ Assay

Serum samples, collected three hours after the first (s.c.) injection, were analyzed for inflammatory cytokines (IFN-α, IFN-β, TNF-α, IL-6, MCP-1 and IL-18) by Mouse Inflammation LEGENDplex™ kit (BioLegend) according to the manufacturer's instructions. Data was acquired on a FACS Verse cytometer (BD Biosciences) and analyzed with BioLegend's LEGENDplex Data Analysis Software. The standard curve regression was used to calculate the concentration of each target cytokine.

### Quantification and Statistical Analysis

Data were analyzed in GraphPad Prism 8 software. All data are presented as mean standard error of the mean (SEM). Data with multiple groups and mean tumor volumes were analyzed by One-way ANOVA with Tukey multiple comparisons test. Survival curves were analyzed by the log-rank (Mantel-Cox) test. p<0.05 was considered significant.

### Results

To optimize the immune response to sub-cutaneous (s.c.) administration of cGAMP-VLPs, the effect of Imatinib on T cells and the tumor growth were investigated.

### Schedule of MCA-OVA Tumor Model

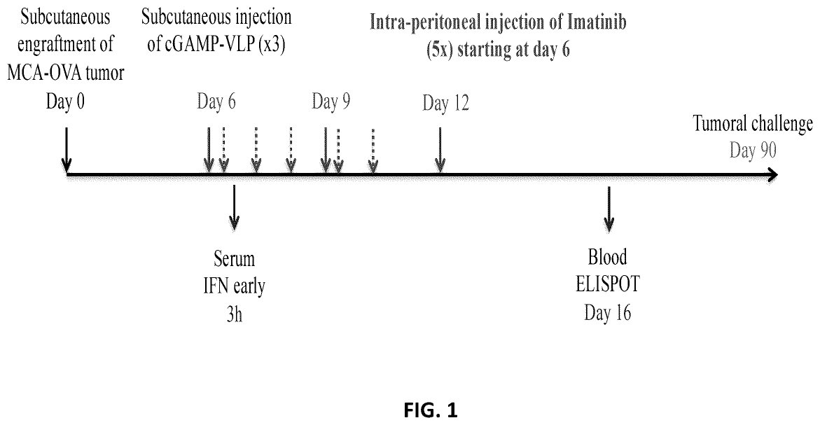

Mice bearing a single MCA-OVA melanoma flank tumor were treated subcutaneously with 50 ng of cGAMP-VLP injected three times over the course of ten days, with or without intraperitoneal injections of Imatinib (10 mg/kg), daily and for five days, starting at day 6 from the tumor engraftment (FIG. 1).

### Synthesis of Inflammatory Cytokines

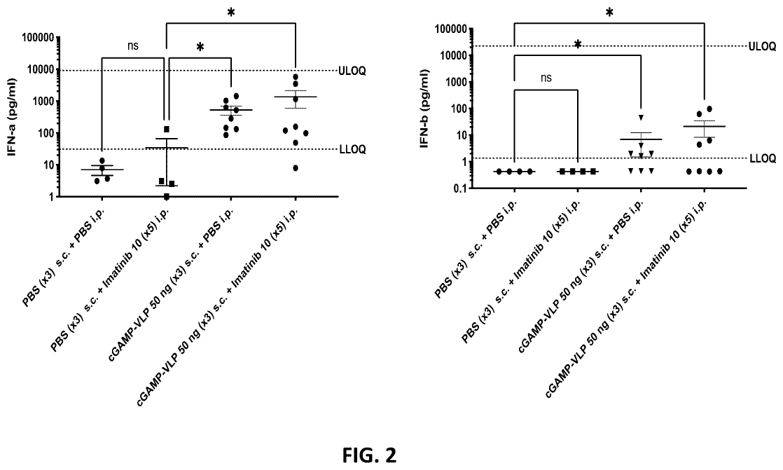

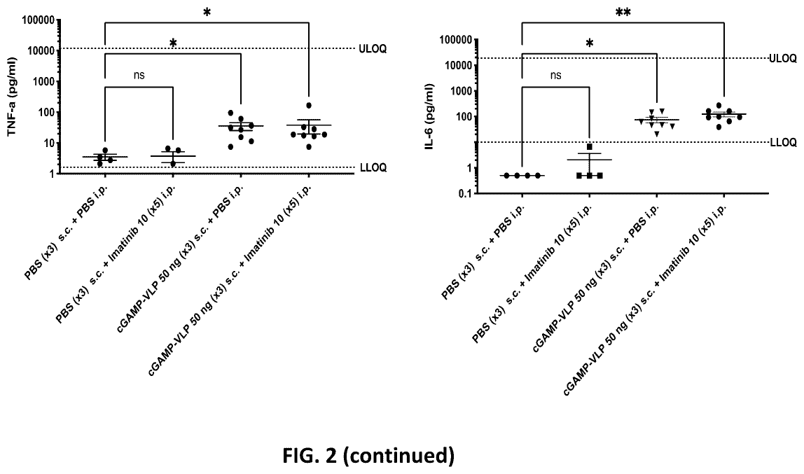

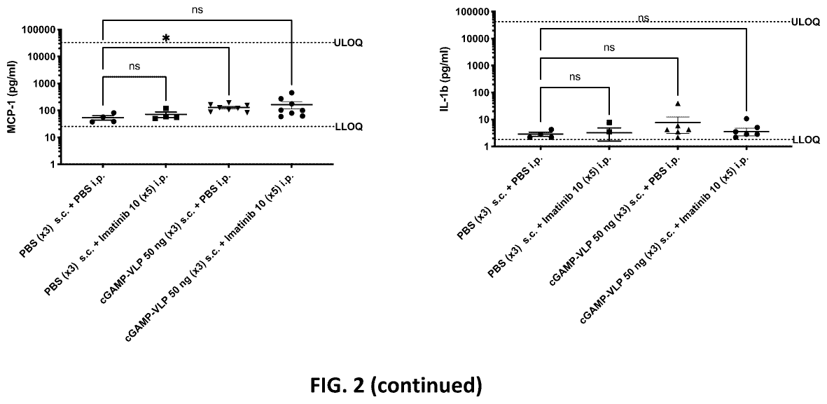

Three hours after the first injection of cGAMP-VLP, the production of inflammatory cytokines was measured in serums by LEGENDplex (FIG. 2). Serums from mice treated with cGAMP-VLP+Imatinib showed a significant increase compared to Imatinib alone and an increase of the cytokine levels (IFN-α, IFN-β, IL-6) comparing to cGAMP-VLP alone.

### CD8 and CD4 T Cell Responses

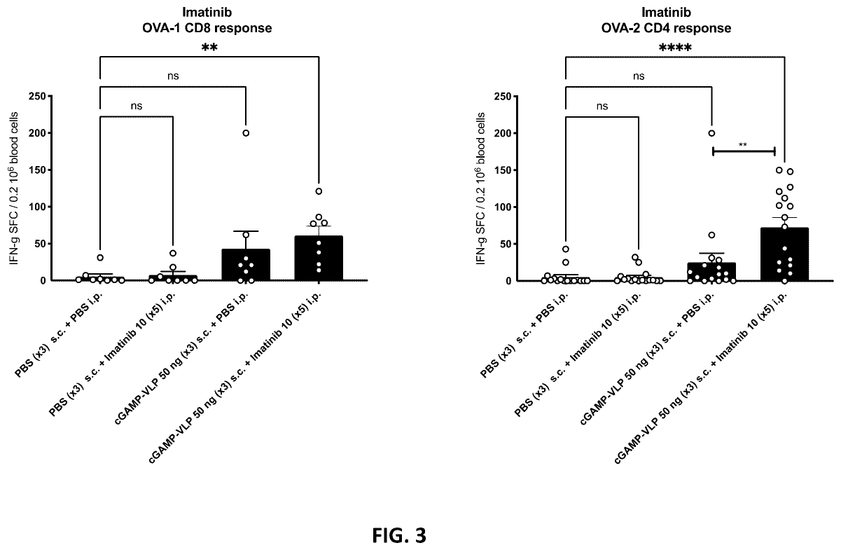

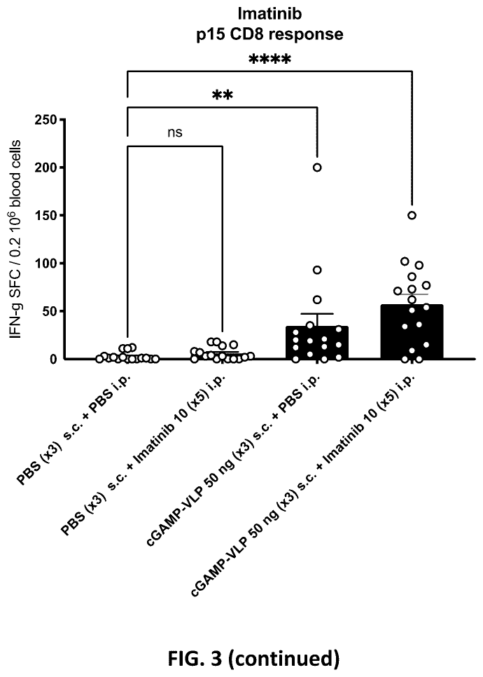

Four days after the third s.c. injection of cGAMP-VLP, the group of mice treated with cGAMP-VLP 7/− Imatinib demonstrated significantly increased tumor-specific T cell responses in peripheral blood compared to the groups treated with PBS 7/− Imatinib (FIG. 3). Imatinib treatment did not induce tumor-specific T cell responses compared to the PBS group. In addition, the group treated with cGAMP-VLP+Imatinib showed higher T-cell responses than cGAMP-VLP alone, which was significant for the OVA-2 peptide.

### Tumor Growth Monitoring

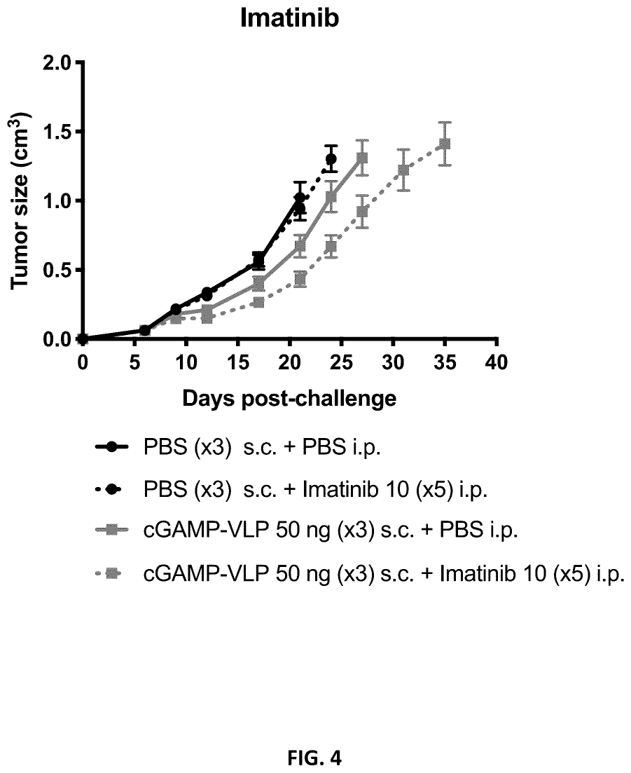

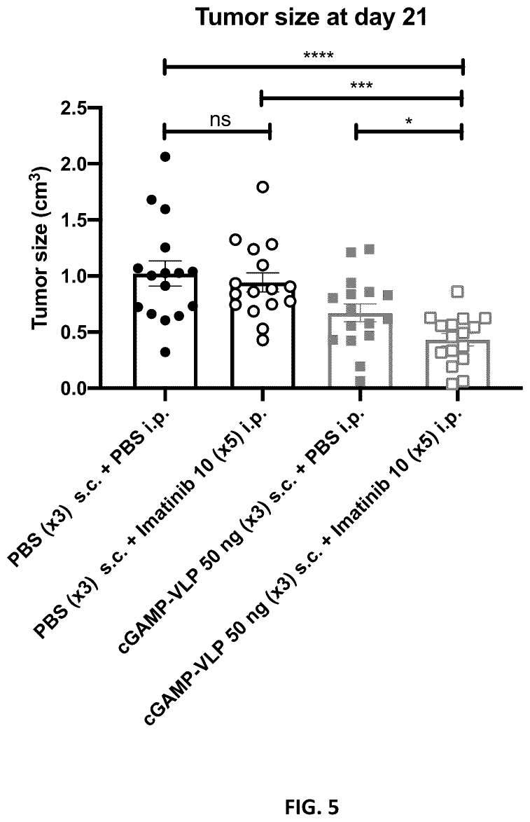

The impact of the treatments on tumor growth was monitored. Imatinib alone had no effect on the tumor growth compared to control group (FIG. 4). cGAMP-VLP injected alone by s.c. route stabilized the tumor growth during a short period with a delay comparing to the control group. The combination of Imatinib treatment with cGAMP-VLP showed a significantly increased anti-tumoral response compared to cGAMP-VLP alone or Imatinib alone (FIG. 5), suggesting a synergistic mechanism of action between cGAMP-VLP and Imatinib.

### Toxicity and Survival of Treated Mice

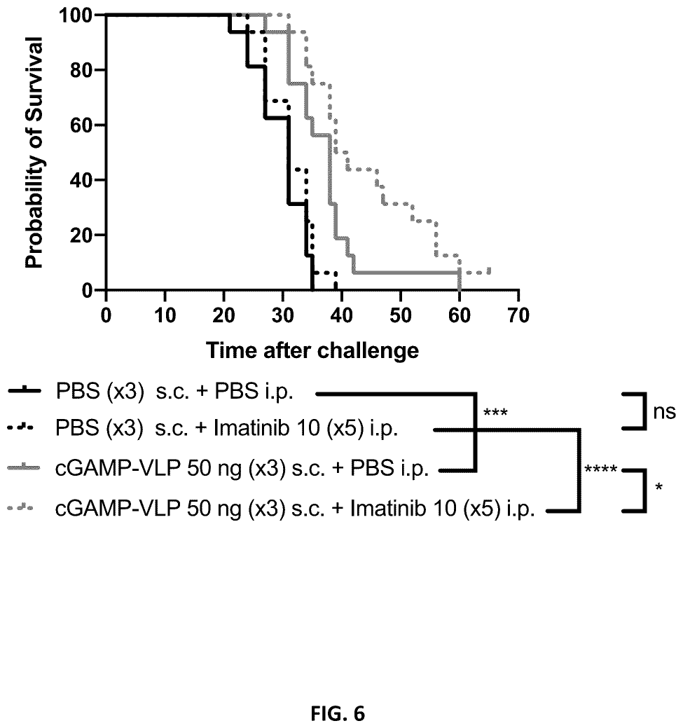

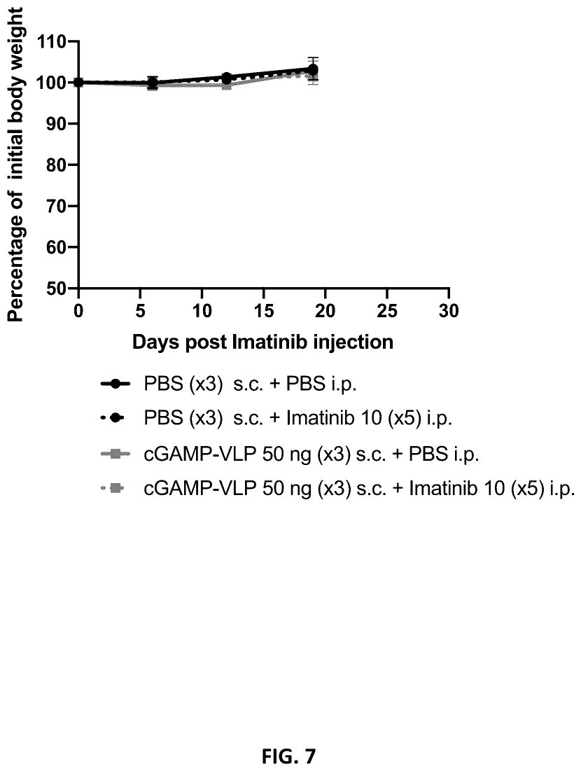

Mice treated Imatinib alone did not show survival benefit compared to PBS treatment (FIG. 6). cGAMP-VLP treatment significantly survival compared to PBS treatment. cGAMP-VLP+Imatinib treatment showed significantly increased survival compared to cGAMP-VLP treatment alone. These results are consistent with a synergistic effect of cGAMP-VLP and Imatinib on protection against tumor-induced death. Mice treated with Imatinib didn't show body weight loss during all the experiment, which confirmed the absence of toxicity with this treatment (FIG. 7).

Inventors conclude that Imatinib treatment demonstrates an unexpected synergy with cGAMP-VLP for generating systemic tumor-specific T cell responses and anti-tumor effects.

### Example No. 2

### Materials and Methods

### Cell Lines

293T cells were cultured in Dulbecco's Modified Eagle Medium (DMEM) supplemented with 10% fetal bovine serum (FBS, GIBCO) and 1% penicillin-streptomycin (GIBCO). THP-1 cells were cultured in Roswell ParkMemorial Institute (RPMI) 1640 medium with 10% FBS (GIBCO) and 1% penicillin-streptomycin (GIBCO). The cells used for the in vivo experiments were the murine fibrosarcoma cell lines MCA-OVA originally purchased from ATCC. Tumor cells were grown in monolayer at 37° C. with 5% CO₂ in RPMI 1640 medium supplemented with 10% fetal calf serum (FCS, Biosera), 1% penicillin-streptomycin and 1 mM 13-Mercaptoethanol (GIBCO). Before reaching confluence, tumor cells were harvested with 0.05% trypsin, washed, and suspended in Hank's Balanced Salt Solution (HBSS, GIBCO) for injection. The cell lines were tested negative for a number of pathogens including Mycoplasma ssp. by PCR.

### Mice

All animals were used according to protocols approved by Animal Committee of Curie Institute and maintained in pathogen-free conditions in a barrier facility. C57BL/6J mice were purchased from Charles River Laboratories. Mice were allowed to acclimate to the housing facility for at least three days. All experiments were initiated using female mice between the ages of 6 and 8 weeks.

cGAMP-VLP Production Before Purification

7.5 million 293T cells are plated in a 150 cm² cell culture flask and incubated overnight. The following day, each flask is transfected with 13 μg of murine cGAS (pVAX1-cGAS), 8.1 μg of HIV-1 GAGPOL (psPAX2), 3.3 μg of VSVG (pVAX1-VSVG-INDIANA2), and 50 μL of PEIpro (Ozyme reference POL115-010), according to the manufacturer's instructions. The transfection mixes were made in Opti-MEM (GIBCO). The morning following transfection, the medium was changed with 52 mL of warm VLP production medium (293T culture medium with added 10 mM HEPES and 50 μg/mL Gentamycin) and the cells were incubated at 37° C. with 5% CO₂ until the following day.

cGAMP-VLP Harvest and Purification on Sucrose Cushion

The cGAMP-VLP containing medium was harvested from the cells, centrifuged for 10 minutes at 200 g at 4° C., and filtered on 0.45 μm. 39 mL of cGAMP-VLP containing medium was gently overlaid on 6 mL of cold 20% sterile filtered endotoxin free sucrose in 6 Ultra-Clear tubes (Beckman Coulter, ref 344058), and centrifuged for 1 hour and 30 minutes at 100′000 g at 4° C. The medium and sucrose was gently aspirated, the pellets were resuspended in cold PBS and transferred to one Ultra-Clear 13.2 mL (Beckman Coulter, ref 344059) and centrifuged again at 100′000 g at 4° C. for 1 hour and 30 minutes. The PBS was gently poured out and the pellet was resuspended in the appropriate amount of cold PBS, typically 320 μL.

2′3′ cGAMP ELISA

After cGAMP extraction with methanol, an ELISA kit was used for quantification of 2′3′-cGAMP in the VLPs according to the manufacturer's instructions (Cayman kit).

### THP-1 Activation and SIGLEC-1 Expression Measure

50′000 THP-1 cells were plated in round bottom 96 well plates in 100 μL of medium, and stimulated with 100 μL of cGAMP-VLPs dilutions and a soluble 2′3′cGAMP dilution. The cells were incubated for 18 to 24 hours and stained with an anti-human SIGLEC-1 (Miltenyi ref 130-098-645), fixed in PFA 1% and acquired using a BD FACS Verse cytometer.

### LEGENDplex™ Assay

Serum samples, collected three hours after the first s.c. injection, were analyzed for inflammatory cytokines (IFN-α, IFN-β, TNF-α, IL-6, MCP-1 and IL-1 B) by Mouse Inflammation LEGENDplex™ kit (BioLegend) according to the manufacturer's instructions. Data was acquired on a FACS Verse cytometer (BD Biosciences) and analyzed with BioLegend's LEGENDplex Data Analysis Software. The standard curve regression was used to calculate the concentration of each target cytokine.

### Tumor Growth

Female C57BL/6J mice were inoculated subcutaneously on the lower right flank with 5×10⁵ MCA-OVA cells in 100 μL of PBS. Mice were monitored for morbidity and mortality daily. Tumors were monitored twice or three times per week. Mice were humanely euthanized if ulceration occurred or when tumor volume reached 2000 mm³. Tumor sizes were measured using a digital caliper and tumor volumes calculated with the formula (length×width²)/2. Following tumor implantation, mice were randomized into treatment groups using the Randmice software powered by Stimunity. Tumor-free survivors were rechallenged with tumor cells on the opposite, non-injected flank several weeks after the collapse of the primary tumor. Naive mice, of the same age, were used as controls.

In vivo Immunotherapy

STING systemic (s.c.) therapy consisted of injecting cGAMP-VLP (50 ng cGAMP per dose) in 50 μL of PBS buffer. S.C. injections were initiated when tumors grew to between 35-50 mm³. A U-100 insulin syringe or equivalent: 0.33 mm (29 G)×12.7 mm (0.5 mL) was filled with the composition and all air bubbles removed. Mice were anesthetized with isoflurane. With the bevel facing the skin, the needle was injected shallowly into the area directly adjacent of the tumor, and the needle was moved underneath the skin until it reached the inside back of the tumor (1 cm). The composition was injected slowly into this area close to the tumor. The needle was then removed delicately to avoid reflux.

In vivo Antibody Treg Depletion

For Treg depletion study, MCA-OVA tumor bearing mice were treated with 200 μg anti-CTLA4 monoclonal antibody (anti-mCTLA4-mIgG2a InvivoFit, Invivogen) or 200 μg isotype control antibody (Mouse IgG2a, BioXcell) on days 6, 9 and 12 by intraperitoneal route (i.p.) after implantation with MCA-OVA melanoma. The Treg depletion was confirmed by FACS using tumors, spleen, draining lymph nodes and blood samples, 48 hours after the last antibodies injection.

Ex vivo Stimulation Assay

T cell responses were assessed by IFN-γ ELISPOT 10 days after the first s.c. injection of cGAMP-VLP or PBS. Mice were bled in the retro-orbital sinus and PBMCs were isolated from whole blood by lysing the red blood cells. 2×10⁵ PBMCs were plated per well in RPMI medium containing 1% penicillin-streptomycin and stimulated overnight with media as a negative control, 10 μg/mL p15 peptide (KSPWFTT:L_, SEQ I) NO: 2), 10 μg/mL OVA 257-264 peptide (OVA-1) (SIIlTEKL, SEQ ID NO: 3) or 40 μg/mL OVA 265-280 peptide (OVA-2) (TEWTSSNVMEERKIKV, SEQ ID NO: 4). Spots were developed using a mouse IFN-γ ELISPOT antibody (Diaclone) according to the manufacturer's instructions, and the number of spots enumerated using an ImmunoSpot analyzer.

### Quantification and Statistical Analysis

Data were analyzed in GraphPad Prism 8 software. All data are presented as mean standard error of the mean (SEM). Data with multiple groups and mean tumor volumes were analyzed by One-way ANOVA with Tukey multiple comparisons test. Survival curves were analyzed by the log-rank (Mantel-Cox) test. p<0.05 was considered significant.

### Results

To study the Treg depletion in tumors and to optimize the immune response to sub-cutaneous (s.c.) administration of cGAMP-VLPs, the effect of the anti-CTLA4-mIgG2a (an isotype that depletes/inhibits Treg specifically in the tumors microenvironment (“TME”), i.e. in and around the tumor but not outside this area, or in other words that does not deplete Treg systemically, for example that does not deplete Treg in blood or in non-draining lymphoid organs) on T cells and the tumor growth were investigated.

### Schedule of MCA-OVA Tumor Model

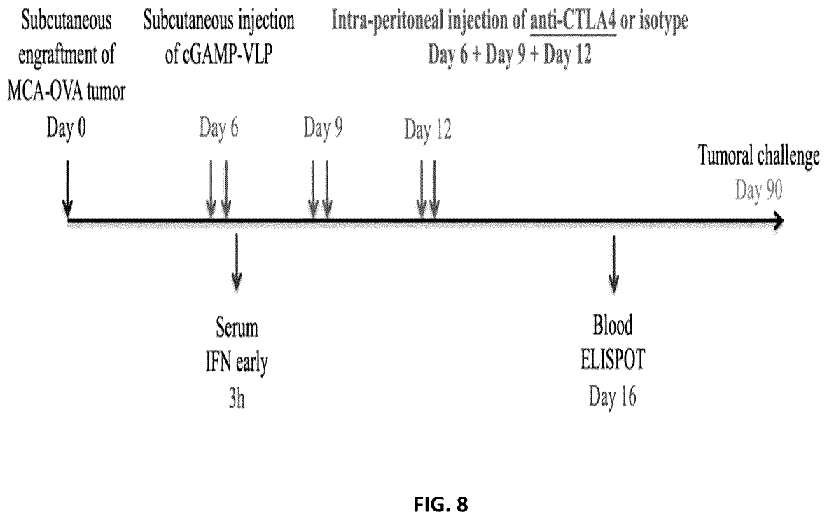

Mice bearing a single MCA-OVA melanoma flank tumor were treated subcutaneously with cGAMP-VLP (50 ng of cGAMP per dose) injected three times over the course of twelve days, with or without intraperitoneal injections of mouse IgG2a anti-CTLA4 (200 μg/mouse), starting at day 6 from the tumor engraftment (FIG. 8).

### Synthesis of Inflammatory Cytokines

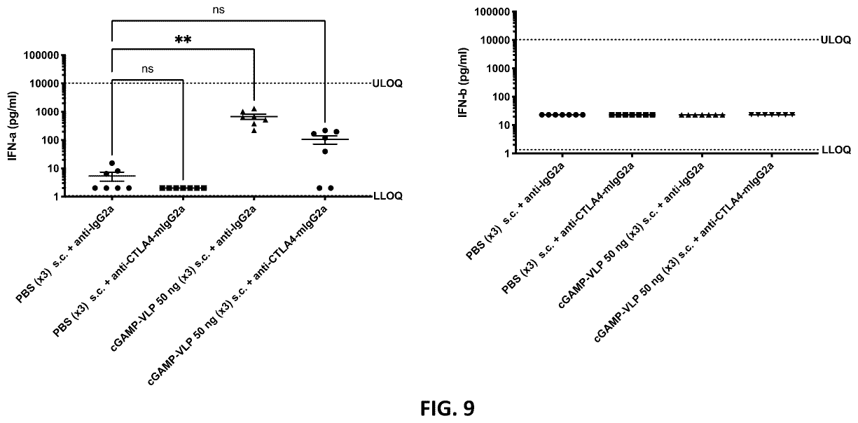

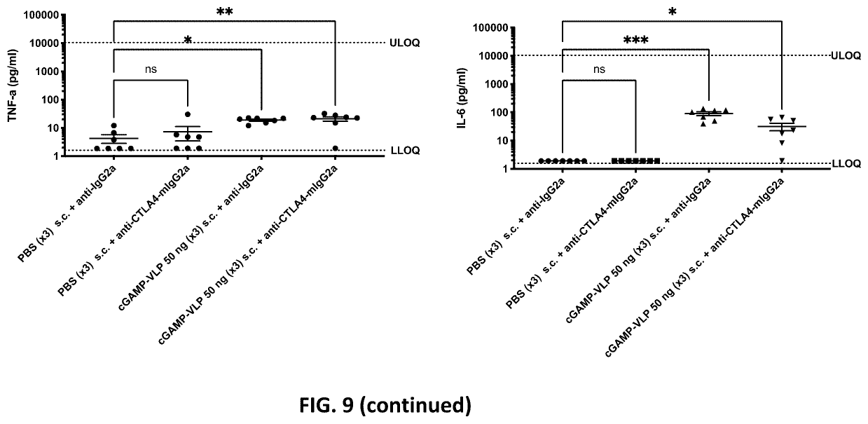

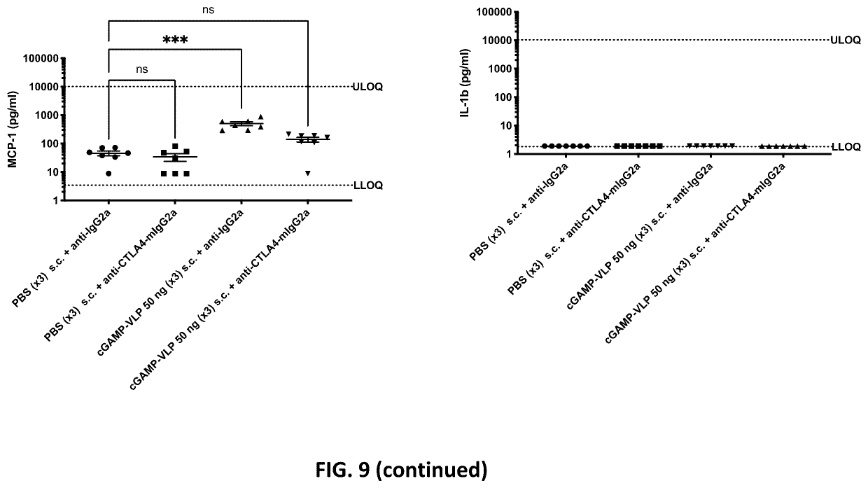

Three hours after the first injection of cGAMP-VLP, the production of inflammatory cytokines was measured in serums by LEGENDplex (FIG. 9). Serums from mice treated with cGAMP-VLP showed a significant increase in the cytokine levels (TL-6 and TNF-α with mIg2a isotype control and anti-CTLA4-mIgG2 groups; IFN-α and MCP-1 only with mIgG2a isotype control group) compared to the groups which received PBS+/−anti-CTLA4-mIgG2a.

### Tregs Depletion

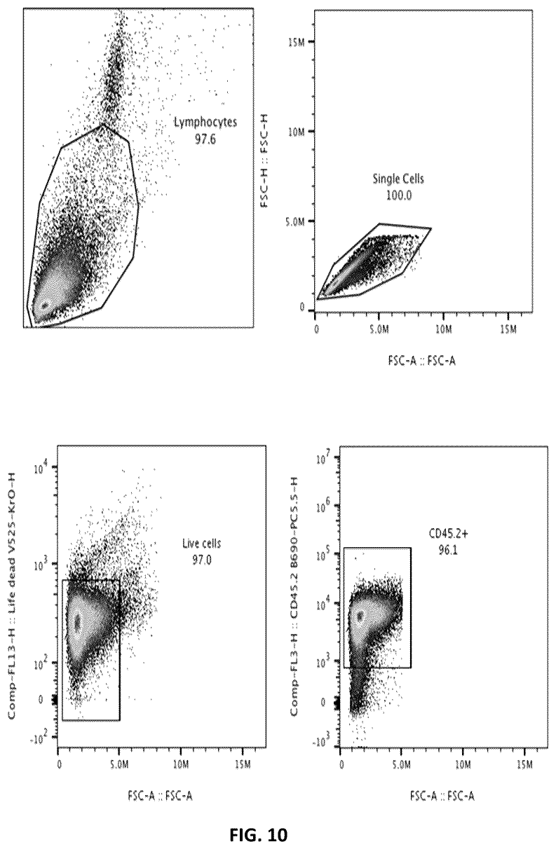

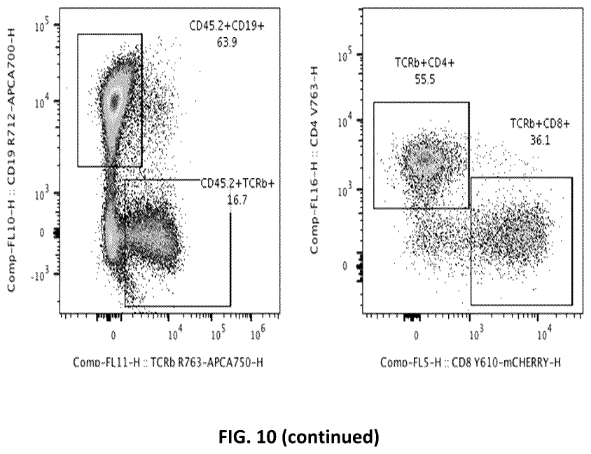

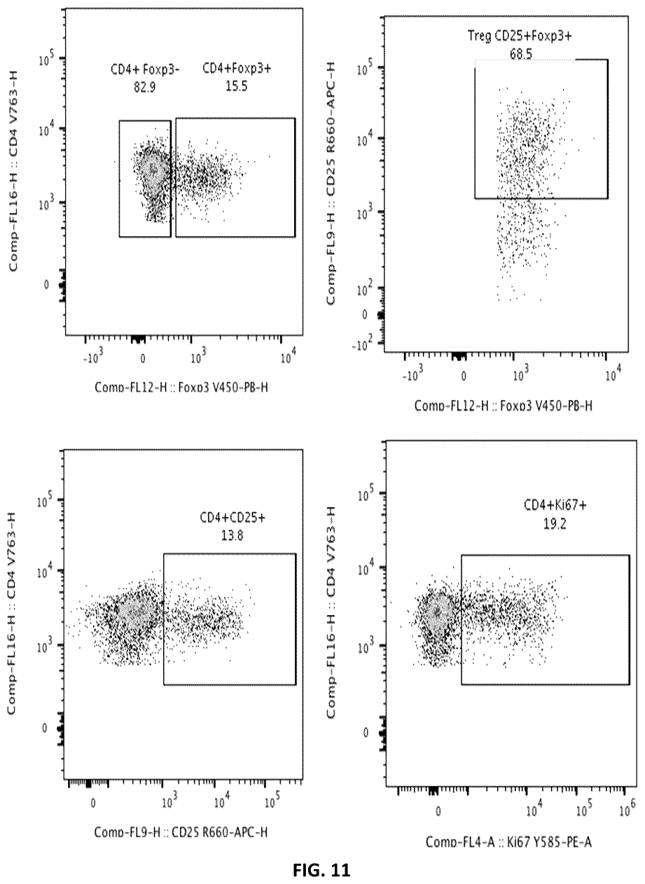

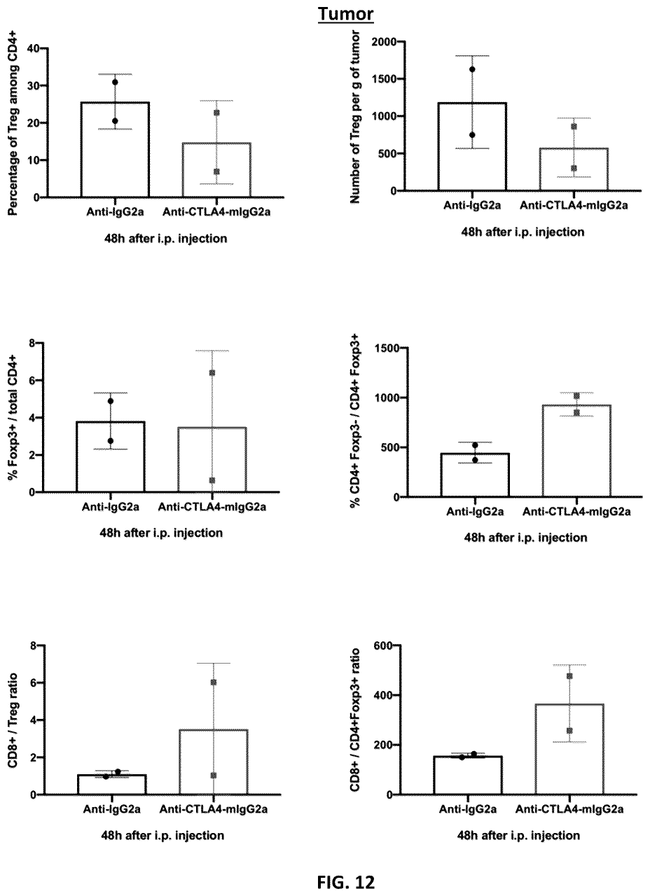

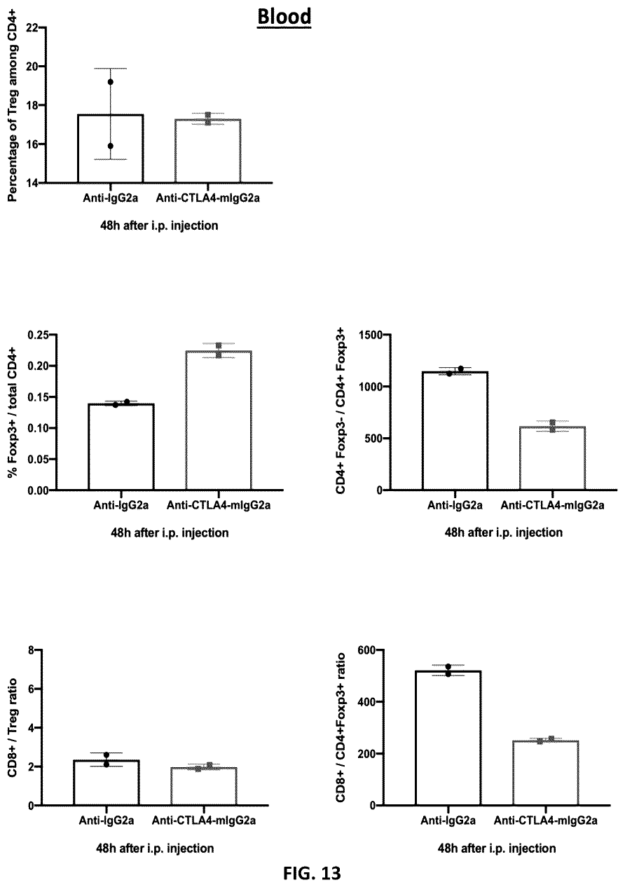

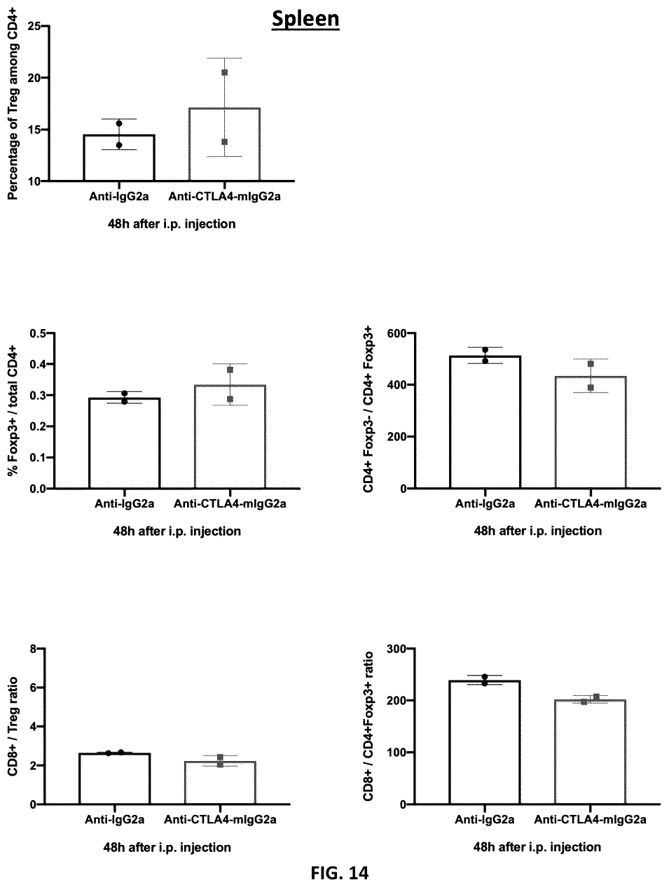

Based on evidence demonstrating the contribution of intra-tumoral Treg cell depletion to the activity of immune modulatory antibodies, inventors compared the impact of anti-CTLA4-mIgG2a on the frequency of Treg cells in the blood, spleen and tumor of mice with established tumors (FIGS. 10 and 11). The administration of 200 μg of anti-CTLA4-mIgG2a on days 6, 9 and 12 after tumor challenge resulted in a reduced frequency of tumor-infiltrating Treg (CD4⁺Foxp3⁺CD25⁺) cells and in an increase of the ratio between CD8⁺ T cells and CD4⁺FoxP3⁺ T cells, and of the ratio between CD4⁺FoxP3⁻ and CD4⁺FoxP3⁺(FIG. 12). However, the anti-CTLA4-mIgG2a failed to deplete the Treg cells in blood (FIG. 13) and spleen (FIG. 14). Their frequency remained comparable to that of untreated mice.

### CD8 and CD4 T Cell Responses

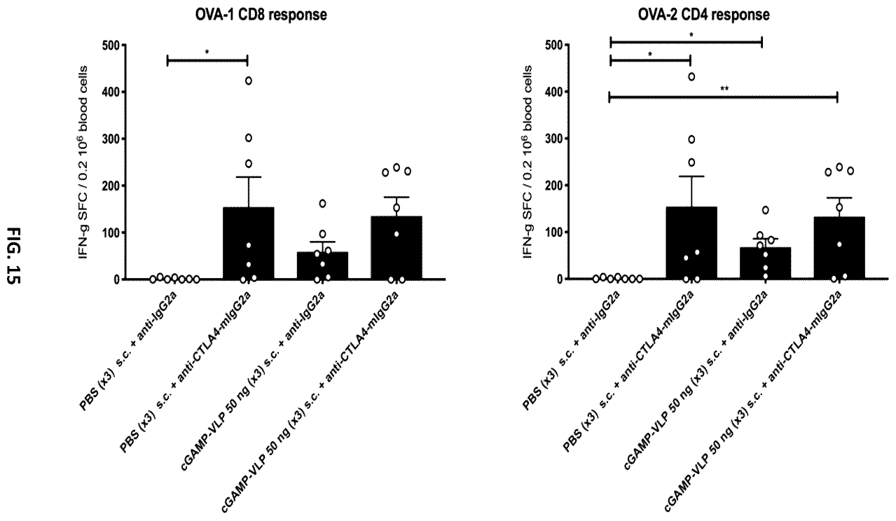

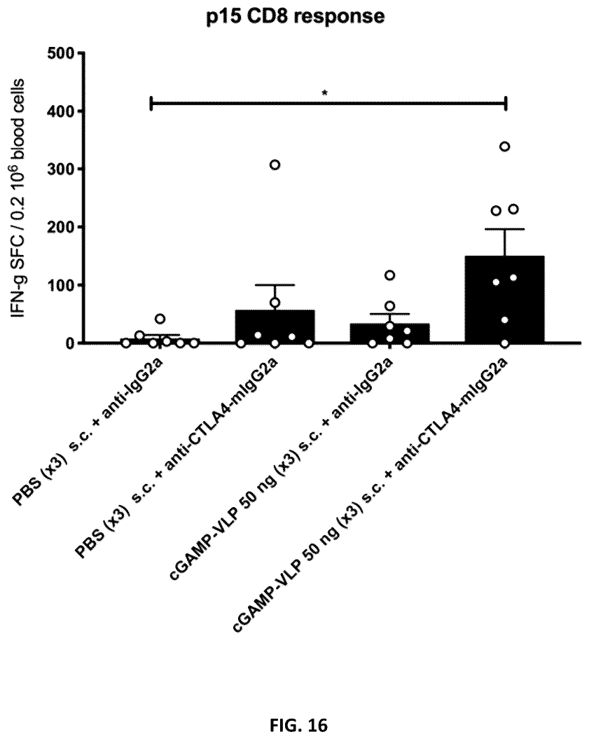

Four days after the third s.c. injection of cGAMP-VLP, the group of mice treated with cGAMP-VLP and/or anti-CTLA4-mIgG2a demonstrated significantly increased OVA-tumor antigen specific T cell responses in peripheral blood compared to the PBS-treated group (FIG. 15). Mice treated with cGAMP-VLP alone or anti-CTLA4-mIgG2a alone did not induce a significant response against p15 tumor antigen. In contrast, combining cGAMP-VLP with anti-CTLA4-mIgG2a induced significant levels of blood T cell response against p15 (FIG. 16).

### Tumor Growth Monitoring

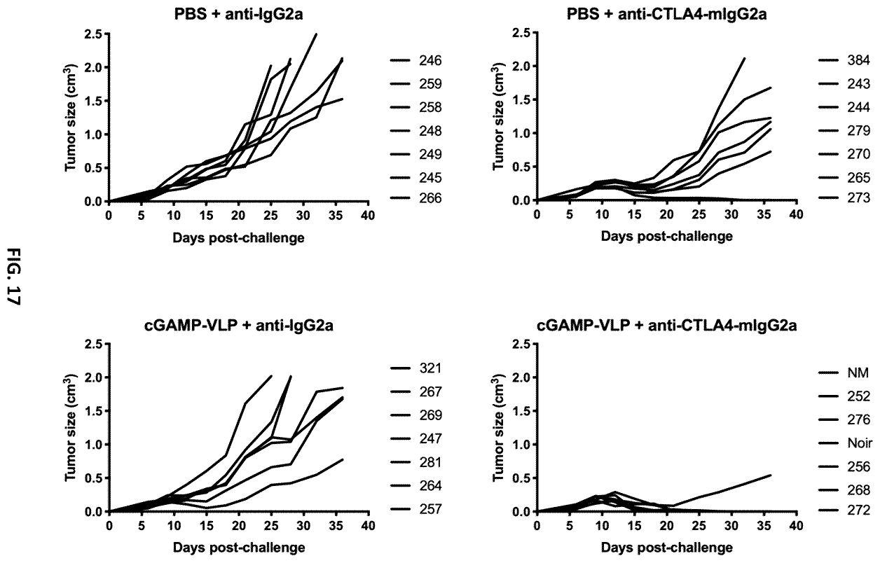

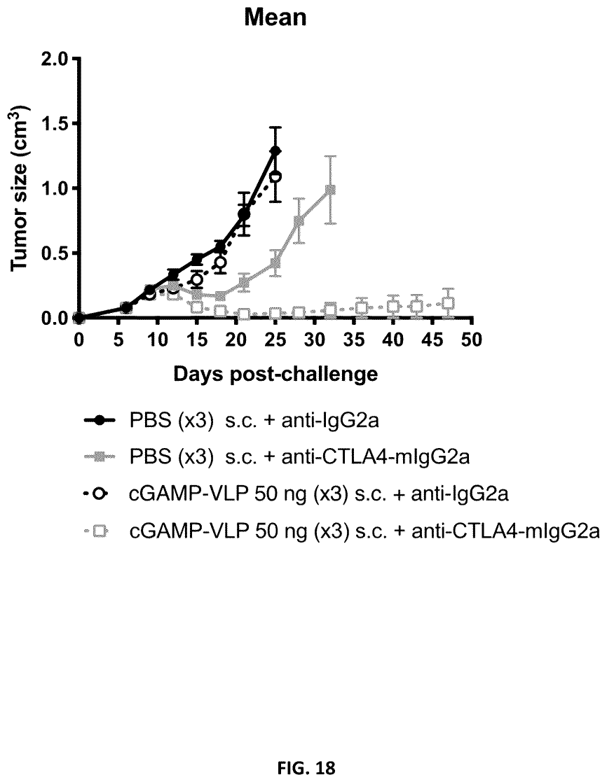

Next, inventors monitored the potential of anti-CTLA4-mIgG2a in treating tumors. Anti-CTLA4-mIgG2a alone was able to induce a stabilization or a decrease of the tumor growth compared to control group (FIG. 17). cGAMP-VLP injected alone by s.c. route stabilized the tumor growth during a short period, with a delay comparing to the control group. However, cGAMP-VLP combined to anti-CTLA4-mIgG2a induced a potent anti-tumoral response and complete tumor regression compared to cGAMP-VLP alone or anti-CTLA4-mIgG2a alone, showing a synergistic mechanism of action between cGAMP-VLP and anti-CTLA4-mIgG2a (FIG. 18).

### Survival of Treated Mice

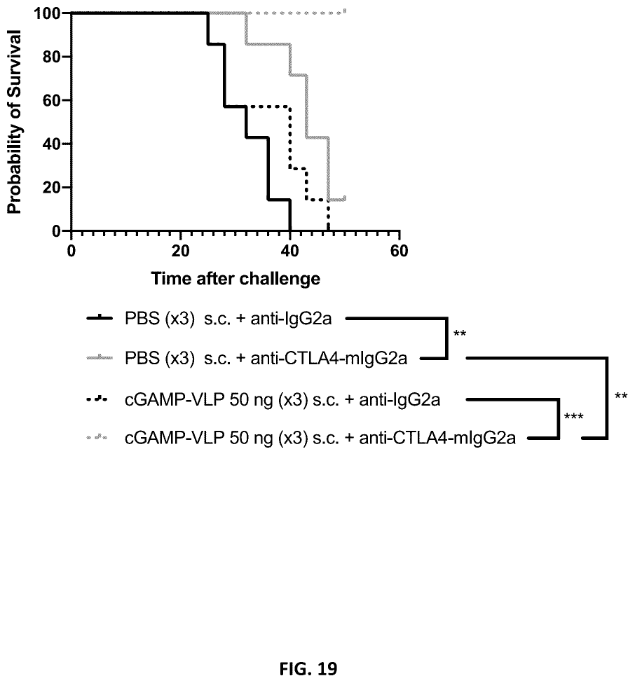

The treatment consisting of cGAMP-VLP with anti-CTLA4-mIgG2a eradicated established MCA-OVA tumors in 100% of the mice, resulting in long-term survival of more than 90 days (FIG. 19).

### Memory Immunity

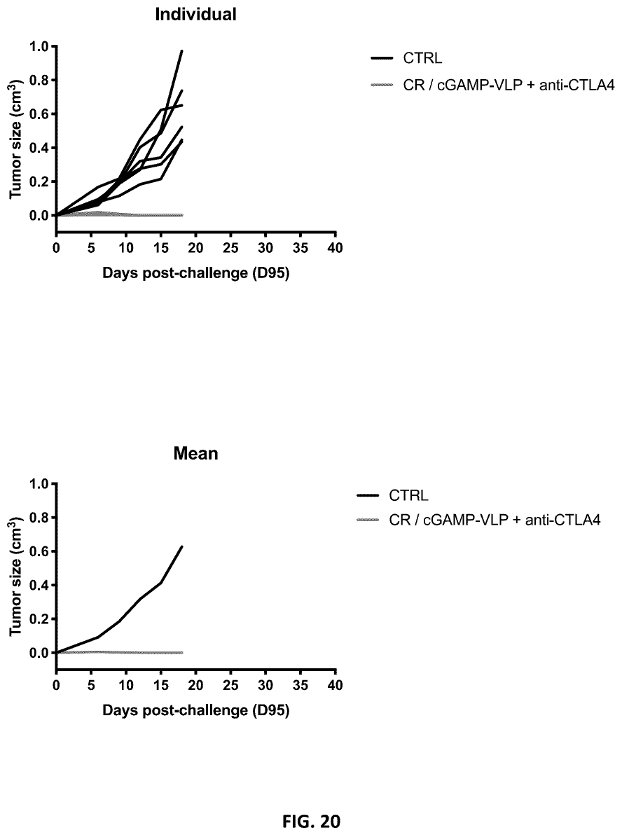

The complete responder mice developed memory T-cell response capable to reject a second MCA-OVA challenge (FIG. 20).

Inventors conclude that anti-CTLA4-mIgG2a results in a synergistic anti-tumor effect when used in combination with cGAMP-VLP, due to the selective depletion of Tregs from the tumor and its micro-environment (i.e., from the TME). The combination generates robust systemic tumor-specific T cell responses and durable anti-tumor and memory immunity.

### Example No. 3

### Materials and Methods

### Cell Lines

293T cells were cultured in Dulbecco's Modified Eagle Medium (DMEM) supplemented with 10% fetal bovine serum (FBS, GIBCO) and 1% penicillin-streptomycin (GIBCO). THP-1 cells were cultured in Roswell ParkMemorial Institute (RPMI) 1640 medium with 10% FBS (GIBCO) and 1% penicillin-streptomycin (GIBCO). The cells used for the in vivo experiments were the murine fibrosarcoma cell lines MCA-OVA originally purchased from ATCC. Tumor cells were grown in monolayer at 37° C. with 5% C02 in RPMI 1640 medium supplemented with 10% fetal calf serum (FCS, Biosera), 1% penicillin-streptomycin and 1 mM 13-Mercaptoethanol (GIBCO). Before reaching confluence, tumor cells were harvested with 0.05% trypsin, washed, and suspended in Hank's Balanced Salt Solution (HBSS, GIBCO) for injection. The cell lines were tested negative for a number of pathogens including Mycoplasma ssp. by PCR.

### Mice

All animals were used according to protocols approved by Animal Committee of Curie Institute and maintained in pathogen-free conditions in a barrier facility. C57BL/6J mice were purchased from Charles River Laboratories. Mice were allowed to acclimate to the housing facility for at least three days. All experiments were initiated using female mice between the ages of 6 and 8 weeks.

cGAMP-VLP Production Before Purification

7.5 million 293T cells are plated in a 150 cm² cell culture flask and incubated overnight. The following day, each flask is transfected with 13 μg of murine cGAS (pVAX1-cGAS), 8.1 μg of HIV-1 GAGPOL (psPAX2), 3.3 μg of VSVG (pVAX1-VSVG-INDIANA2), and 50 μL of PEIpro (Ozyme reference POL115-010), according to the manufacturer's instructions. The transfection mixes were made in Opti-MEM (GIBCO). The morning following transfection, the medium was changed with 52 mL of warm VLP production medium (293T culture medium with added 10 mM HEPES and 50 μg/mL Gentamycin) and the cells were incubated at 37° C. with 5% CO₂ until the following day.

cGAMP-VLP Harvest and Purification on Sucrose Cushion

The cGAMP-VLP containing medium was harvested from the cells, centrifuged for 10 minutes at 200 g 4° C., and filtered on 0.45 μm. 39 mL of cGAMP-VLP containing medium was gently overlaid on 6 mL of cold 20% sterile filtered endotoxin free sucrose in 6 Ultra-Clear tubes (Beckman Coulter, ref 344058), and centrifuged for 1 hour and 30 minutes at 100′000 g at 4° C. The medium and sucrose was gently aspirated, the pellets were resuspended in cold PBS and transferred to one Ultra-Clear 13.2 mL (Beckman Coulter, ref 344059) and centrifuged again at 100′000 g at 4° C. for 1 hour and 30 minutes. The PBS were gently poured out and the pellet was resuspended in the appropriate amount of cold PBS, typically 320 μL.

### THP-1 Activation and SIGLEC-1 Expression Measure

50′000 THP-1 cells were plated in round bottom 96 well plates in 100 μL of medium, and stimulated with 100 μL of cGAMP-VLPs dilutions and a soluble 2′3′cGAMP dilution. The cells were incubated for 18 to 24 hours and stained with an anti-human SIGLEC-1 (Miltenyi ref 130-098-645), fixed in PFA 1% and acquired using a BD FACS Verse cytometer.

### Tumor Growth

Female C57BL/6J mice were inoculated subcutaneously on the lower right flank with 5×10⁵ MCA-OVA cells in 100 μL of PBS. Mice were monitored for body weight loss, morbidity and mortality daily. Tumors were monitored twice or three times per week. Mice were humanely euthanized if ulceration occurred or when tumor volume reached 2000 mm³. Tumor sizes were measured using a digital caliper and tumor volumes calculated with the formula (length×width²)/2. Following tumor implantation, mice were randomized into treatment groups using the Randmice software powered by Stimunity.

In vivo Immunotherapy

STING systemic (subcutaneous, s.c.) therapy consisted of injecting cGAMP-VLP (50 ng cGAMP) in 50 μL of PBS buffer. S.C. injections were initiated when tumors grew to between 35-50 mm³. A U-100 insulin syringe or equivalent: 0.33 mm (29 G)×12.7 mm (0.5 mL) was filled with the composition and all air bubbles removed. Mice were anesthetized with isoflurane. With the bevel facing the skin, the needle was injected shallowly into the area directly adjacent of the tumor, and the needle was moved underneath the skin until it reached close to the tumor (1 cm). The composition was injected slowly into this area close to the tumor. The needle was then removed delicately to avoid reflux.

In vivo Antibody Treg Depletion

For Treg depletion study, MCA-OVA tumor bearing mice were treated with 150 μg anti-CD25 IgG2a (KLC) monoclonal antibody (mIgG2a KLC Anti-mCD25, Invivogen) or 150 μg isotype control antibody (mouse anti-b-Gal-mIgG2a, Invivogen) on days 6, 9 and 12 by intraperitoneal route (i.p.) after implantation with MCA-OVA melanoma. The Treg depletion was confirmed by FACS using tumors and spleen samples, 48 hours after the last antibodies injection.

Ex vivo Stimulation Assay

T cell responses were assessed by IFN-γ ELISPOT 10 days after the first s.c. injection of cGAMP-VLP or PBS. Mice were bled in the retro-orbital sinus and PBMCs were isolated from whole blood by lysing the red blood cells. 2×10⁵ PBMCs were plated per well in RPMI medium containing 1% penicillin-streptomycin and stimulated overnight with media as a negative control, 10 μg/mL OVA 257-264 peptide (OVA-1) (SIIN FEKL, SEQ ID NO: 3) or 40 μg/mL OVA 265-280 peptide (OVA-2) (TEWTSSNVMEERKIKV, SEQ ID NO: 4). Spots were developed using a mouse IFN-γ ELISPOT antibody (Diaclone) according to the manufacturer's instructions, and the number of spots enumerated using an ImmunoSpot analyzer.

### Quantification and Statistical Analysis

Data were analyzed in GraphPad Prism 8 software. All data are presented as mean standard error of the mean (SEM). Data with multiple groups and mean tumor volumes were analyzed by One-way ANOVA with Tukey multiple comparisons test. Survival curves were analyzed by the log-rank (Mantel-Cox) test. p<0.05 was considered significant.

### Results

To study the Treg depletion in tumors and to optimize the immune response to sub-cutaneous (s.c.) administration of cGAMP-VLPs, the effect of the anti-CD25-mIgG2a (an isotype that depletes/inhibits Treg specifically in the tumor microenvironment (“TME”), i.e. in and around the tumor but not outside this area, or in other words that does not deplete Treg systemically, for example that does not deplete Treg in blood or in non-draining lymphoid organs) on T cells and the tumor growth were investigated.

### Schedule of MCA-OVA Tumor Model

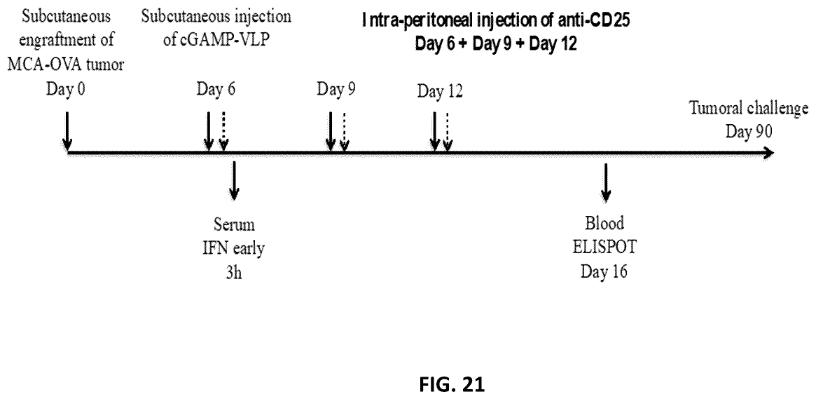

Mice bearing a single MCA-OVA melanoma flank tumor were treated subcutaneously with cGAMP-VLP (50 ng of cGAMP per dose) injected three times over the course of twelve days, with or without intraperitoneal injections of mouse IgG2a anti-CD25 (150 μg/mouse), starting at day 6 from the tumor engraftment (FIG. 21).

### Tregs Depletion

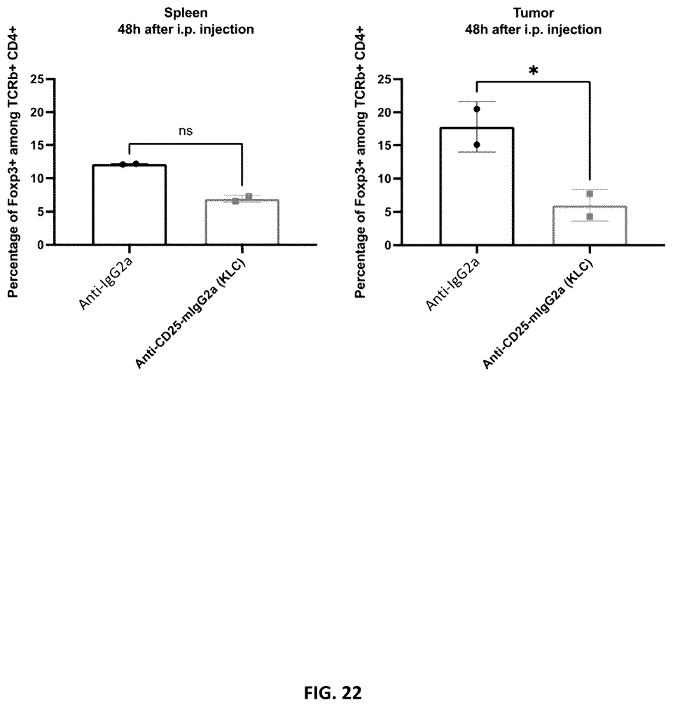

Based on evidence demonstrating the contribution of intra-tumoral Treg cell depletion to the activity of immune modulatory antibodies, inventors compared the impact of anti-CD25-mIgG2a on the frequency of Treg cells in spleen and tumor of mice with established tumors (FIG. 22). The administration of 150 μg of anti-CD25-mIgG2a on days 6, 9 and 12 after tumor challenge resulted in a statistically significant reduced frequency of tumor-infiltrating FOXP3m cells (among total TCRb⁺CD4⁺ cells). However, the anti-CD25-mIgG2a slightly and non-significantly depleted the FOXP3⁺ cells in spleen. Their frequency remained comparable to that of untreated mice.

### CD8 and CD4 T Cell Responses

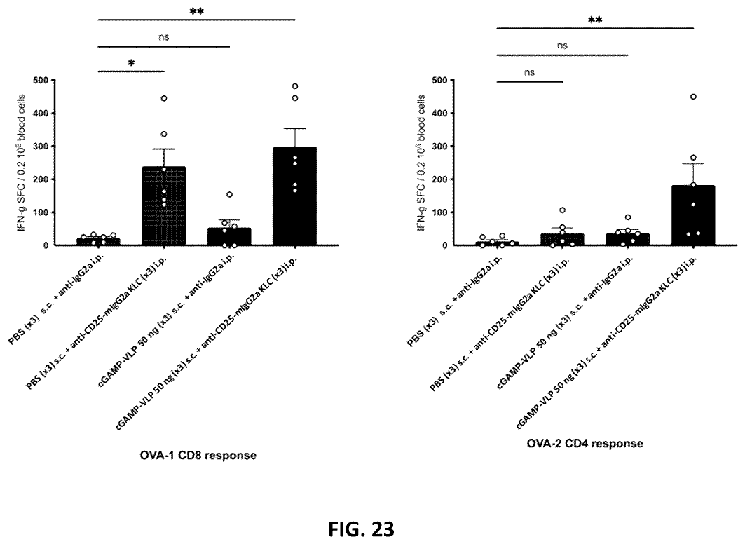

Four days after the third s.c. injection of cGAMP-VLP, the group of mice treated with cGAMP-VLP demonstrated slightly increased OVA-tumor antigen specific T cell responses in peripheral blood compared to the PBS-treated group (FIG. 23). Mice treated with anti-CD25-mIgG2a alone did not induce a significant response against OVA 265-280 tumor antigen. In contrast, combining cGAMP-VLP with anti-CD25-mIgG2a induced significant levels of blood CD4 T cell response against OVA peptides.

### Tumor Growth Monitoring

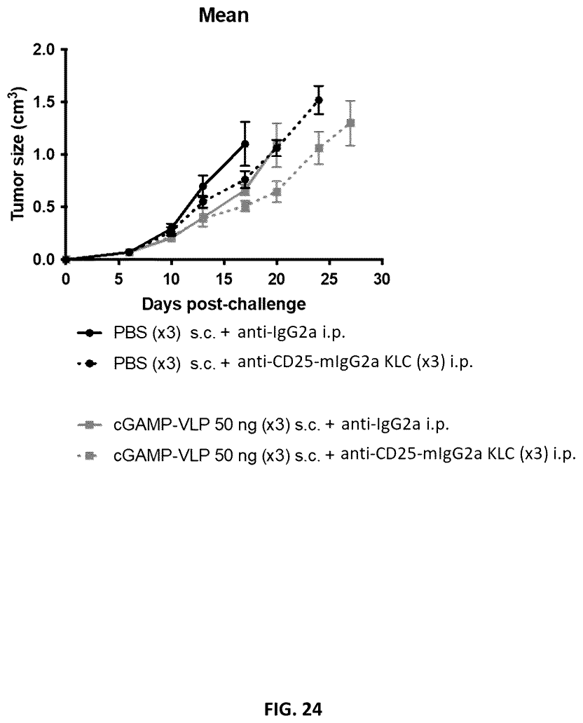

Next, inventors monitored the potential of anti-CD25-mIgG2a in treating tumors. Anti-CD25-mIgG2a alone was able to induce a stabilization or a decrease of the tumor growth compared to control group (FIG. 24). cGAMP-VLP injected alone by s.c. route stabilized the tumor growth during a short period, with a delay comparing to the control group. However, cGAMP-VLP combined to anti-CD25-mIgG2a induced a significant anti-tumoral response and delay in tumor growth compared to PBS control group, showing a synergistic mechanism of action between cGAMP-VLP and anti-CD25-mIgG2a. The treatment consisting of cGAMP-VLP combined to anti-CD25-mIgG2a declined established MCA-OVA tumors in mice, resulting in a delay of mice death for 10 more days comparing to the control group.

Inventors conclude that anti-CD25-mIgG2a results in a synergistic anti-tumor effect when used in combination with cGAMP-VLP, due to the selective depletion/inhibition of Tregs from the tumor and its microenvironment (i.e., from the TME). The combination generates robust systemic tumor-specific T cell responses and anti-tumor responses.

1-15. (canceled)

16. A pharmaceutical combination comprising i) a vector comprising a stimulator of interferon genes (STING) activator and ii) an anti-regulatory T cells (Tregs) agent selected from imatinib or a derivative or salt thereof; dasatinib or a derivative or salt thereof; an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof.

17. The combination according to claim 16, wherein the STING activator is selected from a small molecule, cyclic dinucleotide; a nucleic acid coding for a STING agonist; a vector or cell expressing STING or a STING agonist; an antibody conjugated STING agonist; and an inhibitor of ectonucleotide pyrophosphatase phosphodiesterase 1 (ENPP1).

18. The combination according to claim 17, wherein the cyclic dinucleotide is cyclic guanosine monophosphate-adenosine monophosphate (cGAMP), cyclic dimeric guanosine monophosphate (c-di-GMP) or cyclic dimeric adenosine monophosphate (c-di-AMP).

19. The combination according to claim 16, wherein the vector is a vesicle, a liposome; an exosome; a virus, an adenovirus, an oncolytic virus; a virus-like particle (VLP); a polymer; or a hydrogel.

20. The combination according to claim 19, wherein the VLP comprises a lipoprotein envelope including a viral fusogenic glycoprotein, and contains cGAMP, the cGAMP being packaged into said virus-like particle.

21. The combination according to claim 17, wherein the STING activator is a small molecule selected from dithio-(RP, RP)-[cyclic[A(2′,5′)pA(3′,5′)p]] (ML RR-S2 CDA; ADU-S100); MK-1454; 5,6-dimethyl-xanthenone acetic acid (DMXAA); Flavone-8-acetic acid (FAA); 10-carboxymethyl-9-acridanone (CMA); α-Mangostin (xanthone); ML RR-S2 CDG; MVL RR-S2 cGAMP; 6-bromo-N-(naphthalen-1-yl)benzo[d][1,3]dioxole-5-carboxamide (BNBC); dispiro diketopiperzine (DSDP); 2′3′-cG^{S}A^{S}MP; a macrocycle-bridged STING agonist (MBSA); E7766; a benzothiophene oxobutanoic acid (MSA-2) derivative; SB 11285; diamidobenzimidazole (diABZI); IMSA-101, TAK-676; CRD5500; TTI-10001; and SEL312-4787.

22. The combination according to claim 16, wherein the mutation of the IgG1 constant region of the anti-CTLA4 antibody is located in a CH2 domain of the (Fc) constant region.

23. The combination according to claim 22, wherein the mutation occurs at an amino acid position selected from 233, 234, 235, 236, 237, 238, 239, 243, 247, 256, 262, 267, 268, 270, 271, 280, 290, 292, 298, 300, 305, 324, 326, 328, 330, 332, 333, 334, 339 or 396 (EU index).

24. The combination according to claim 23, wherein each of the two CH2 domains of the Fc constant region comprises at least one mutation at an amino acid position selected from 239, 330 and 332.

25. The combination according to claim 24, wherein each of the two CH2 domains of the Fc constant region comprises S239D, A330L and/or 1332E substitutions.

26. The combination according to claim 23, wherein the first CH2 domain of the Fc constant region comprises at least one mutation at an amino acid position selected from 234, 235, 236, 239, 268, 270, 298, and the second CH2 domain of the Fc constant region comprises at least one mutation at an amino acid position selected from 270, 326, 330 and 334.

27. The combination according to claim 26, wherein the first CH2 domain of the Fc constant region comprises a substitution selected from L234Y, L235Q, G236W, S239M, H268D, D270E and S298A.

28. The combination according to claim 25, wherein the second CH2 domain of the Fc constant region comprises a substitution selected from D270E, K326D, A330M and K334E.

29. A method for treating cancer in a subject comprising administering a pharmaceutical combination comprising i) a vector comprising a stimulator of interferon genes (STING) activator and ii) an anti-regulatory T cells (Tregs) agent selected from imatinib or a derivative or salt thereof; dasatinib or a derivative or salt thereof; an anti-CTLA-4 antibody having an IgG2 constant region or a mutated IgG1 constant region; an anti-LAG3 antibody; an anti-TIM-3 antibody; an anti-ICOS antibody; an anti-CD25 antibody; an anti-CCR4 antibody; an anti-CCR8 antibody; an anti-TNFR2 antibody; and any combination thereof.

30. The method according to claim 29, wherein the STING activator is to be administered to the subject by subcutaneous, oral, intraperitoneal, intramuscular, intradermal or intravenous route.

31. The method according to claim 29, wherein the anti-Tregs agent is to be administered to the subject by intraperitoneal, oral, subcutaneous or intravenous route.

32. A kit comprising at least two parts, wherein the first part comprises a vector comprising a stimulator of interferon genes (STING) activator and the second part comprises an anti-regulatory T cells (Tregs) agent, the first and second parts of the kit being in distinct compartments or containers.

33. The kit according to claim 32, wherein the stimulator of interferon genes (STING) activator is a virus-like particle (VLP) comprising a lipoprotein envelope including a viral fusogenic glycoprotein, and contains cyclic guanosine monophosphate-adenosine monophosphate (cGAMP), the cGAMP being packaged into said virus-like particle, and the anti-regulatory T cells (Tregs) agent is either imatinib or a derivative or salt thereof, or an anti-CTLA-4 antibody having an IgG2 constant region or a mutated IgG1 constant region.
